(19) United States
(12) Patent Application Publication
(10) Pub. No.: US 2016/0228633 A1
(43) Pub. Date: Aug. 11, 2016

(54) INFUSION PUMP WITH TOUCHLESS USER INTERFACE AND RELATED METHODS

(71) Applicant: Smiths Medical ASD, Inc., Plymouth, MN (US)

(72) Inventors: Michael D. Welsch, Stillwater, MN (US); Christopher Allen Lacy, Arden Hills, MN (US); Larry R. Zalesky, Shoreview, MN (US); Grant A. Adams, Coon Rapids, MN (US); Michael Ruby, Plymouth, MN (US); Eric Wilkowske, North Oaks, MN (US); Jim Drost, Woodbury, MN (US); Chris Quinn, Plymouth, MN (US)

(73) Assignee: Smiths Medical ASD, Inc., Plymouth, MN (US)

(21) Appl. No.: 15/025,054

(22) PCT Filed: Aug. 19, 2014
(86) PCT No.: PCT/US14/51614

§ 371 Date: Mar. 25, 2016

Related U.S. Application Data
(60) Provisional application No. 61/883,569, filed on Sep. 27, 2013.

(51) Int. Cl. A61M 5/142; G06F 3/03; G06K 9/18; G06F 3/044; G06F 3/0488; G06K 9/00; G06F 3/01; G06F 3/16

(52) U.S. Cl. CPC A61M 5/142; G06F 3/017; G06F 3/0304; G06F 3/167; G06F 3/044; G06F 3/015; G06F 3/0488; G06K 9/00228; G06K 9/18; A61M 2005/14208; A61M 2205/609; A61M 2205/3317; A61M 2205/80; A61M 2205/6009; A61M 2205/6054; G06F 2203/04108

(57) ABSTRACT

Embodiments include an infusion pump providing safe and reliable touchless control. The infusion pump includes a pump housing, a pumping mechanism coupled to the pump housing that selectively delivers medicament to a patient, a pump control system including a processor and a memory programmable to control operation of the pumping mechanism, and a touchless control module for relaying commands to the pump control system. The touchless control module includes a first touchless user interface configured to receive a touchless programming command for the infusion pump from a user and a second touchless user interface configured to confirm the touchless programming command by receipt of a touchless confirmation command from the user.

### RELATED APPLICATION

This application claims priority to U.S. Provisional Patent Application No. 61/883,569 filed Sep. 27, 2013, which is incorporated herein by reference in its entirety.

### TECHNICAL FIELD

Embodiments relate generally to an infusion pump having a touchless interface that can be programmed and operated without physical operator contact with the infusion pump and related methods. Embodiments of the infusion pump further utilize methods for accurate programming and touchless verification features to ensure safe delivery of fluids, nutrients and medications (collectively, “medicaments”) to patients.

### BACKGROUND

Infusion pumps are extremely useful medical devices for providing prescribed medicaments and drug therapies to patients. For example, medications such as antibiotics, chemotherapy drugs, and pain relievers are commonly delivered to patients via an infusion pump. Infusion pumps have been used in hospitals, nursing homes, and in other short-term and long-term medical facilities, as well as for in-home care. Infusion pumps are particularly useful for the delivery of medical therapies requiring an extended period of time for their administration. There are many types of infusion pumps, including large volume, patient-controlled analgesia (PCA), peristaltic, elastomeric, syringe, enteral, and insulin pumps. Infusion pumps are typically useful in various routes of medication delivery, including intravenously, intra-arterially, subcutaneously, intraperitoneally, in close proximity to nerves, and into an intraoperative site, epidural space or subarachnoid space. Currently, most infusion pumps are locally controlled via the programming of the individual infusion pump. Clinicians and patients rely on infusion pumps for safe and accurate administration of medicaments.

Patient safety has always been paramount in hospitals and medical environments generally. This has been especially true when dealing with vulnerable patients and situations in which potent medications capable of causing significant physiological or chemical effects are being administered. Accordingly, medical practitioners strive to ensure that patients receive safe and appropriate medical care including appropriate infusions of medicaments. The “five rights of medication administration” (also referred to as “the five rights” in this application) commonly referenced in connection with ensuring safe infusions, are: right patient (for example, determining that the medicament was prescribed for the correct patient), right drug or medication (for example, determining that a particular medicament has been prescribed correctly), right dose (for example, determining that the correct volume or number of milliliters, tablets, or doses of the medicament are to be given to the patient), right route (for example, determining that it is correct that the medicament is given to the patient intravenously or by mouth, feeding tube, or other injection, etc.) and right time (for example, determining that the medicament is delivered to the patient at the correct time of day). With these “rights” in mind, a constant aim for infusion pumps has been increased safety and ensuring these “rights” are kept. Infusion pump manufacturers and users have been keenly interested in ensuring that these “five rights” are implemented, observed, and verified. Improvements in infusion pumps that can in turn improve patient safety continue to be desired by the medical community. In addition to addressing safety issues involving infusion pumps specifically, improvements in medical practices are continuously desired to help mitigate general safety concerns involving lack of hygiene and cleanliness of medical equipment.

Improved systems and methods are desired to provide better care to patients in need of medicaments. Therefore, improved infusion pumps and methods, that provide increased patient safety and which are conducive to promoting a clean patient environment, are desired. In this regard, a touchless user interface system and method would be distinctly advantageous.

### SUMMARY

Embodiments relate to an infusion pump providing safe and reliable touchless control. The infusion pump includes a pump housing, a pumping mechanism coupled to the pump housing that selectively urges medicament along an infusion line to a patient (or otherwise deliver medicament to the patient), a pump control system including a processor and a memory programmable to control operation of the pumping mechanism, and a touchless control module for relaying commands to the pump control system. The touchless control module includes a first touchless user interface configured to receive a touchless programming command for the infusion pump from a user and a second touchless user interface configured to confirm the touchless programming command by receipt of a touchless confirmation command from the user.

Another embodiment is directed to an infusion pump providing safe and reliable touchless control. Specifically, the infusion pump includes a voice-based touchless user interface that receives voice commands requesting one or more modifications of operating parameters of the infusion pump and a gesture-based touchless user interface that receives user confirmation of the one or more modifications of operating parameters of the infusion pump.

A further embodiment relates to a method of safely and reliably controlling an infusion pump in a touchless manner. The method includes programming an infusion pump with one or more commands using a first type of touchless input in response to one or more touchless, gesture-based movements received by a touchless sensing module in the infusion pump and verifying the one or more commands with a second type of touchless input received by the touchless sensing module in the infusion pump. In some embodiments, this method can use multiple touchless user interfaces to provide safe medicament delivery in an infusion pump.

A further embodiment relates to a method of safe medicament delivery in an infusion pump, including receiving a programming change in an infusion pump and using two touchless user interfaces relying on different types of touchless sensing technology to control programming changes to the infusion pump. In this method, one of the touchless user interfaces confirms the commands made by the other touchless user interface.

Another embodiment is directed to an infusion pump providing safe and reliable control. The infusion pump includes a pump housing and a pumping mechanism coupled to the pump housing that selectively urges medicament along an infusion line to a patient (or otherwise deliver medicament to the patient). The pump also includes a pump control system including a processor and a memory programmable to control operation of the pumping mechanism. Further, the infusion pump includes an authentication system of an infusion pump. The authentication system includes a user interface associated with the infusion pump having an input device that recognizes and logs at least one user identifier associated with a user of the infusion pump, and an authentication module that authorizes the user to control the infusion pump upon recognition of the at least one user identifier as being associated with an authorized user of the infusion pump.

An embodiment relates to a method of safely and reliably controlling an infusion pump including authenticating an authorized user of an infusion pump. Authenticating an authorized user can include requesting a user identifier including information from a potential user and determining whether the potential user meets the requirements of authorized users of the infusion pump based upon the user identifier received in response to the request. Embodiments can further include receiving a programming of a delivery of medicament to a patient via the infusion pump from the authorized user and authenticating the programming of a delivery of medicament to a patient via the infusion pump. Authenticating the programming can include requesting confirmation of the patient programmed to receive the medicament from the infusion pump, requesting confirmation of the medicament programmed to be delivered by the infusion pump, requesting confirmation of a dose programmed to be delivered by the infusion pump, requesting confirmation of a route of programmed delivery of the infusion pump, requesting confirmation of a time of programmed medicament delivery by the infusion pump, and determining whether the programming of a delivery of medicament is authorized based on responses of the authorized user to the requested confirmations.

Further embodiments relate to identification and authentication systems for authorized practitioners or users of infusion pumps, which may also be provided in combination with “five rights” verification capabilities to further enhance overall patient safety.

An embodiment relates to a further infusion pump providing safe and reliable touchless control including a pump housing, a pumping mechanism coupled to the pump housing that selectively urges medicament along an infusion line to a patient (or otherwise deliver medicament to the patient), and a pump control system including a processor and a memory programmable to control operation of the pumping mechanism. The infusion pump further including a touchless control module for relaying commands to the pump control system having a first touchless proximity sensor configured to receive a touchless command for the infusion pump from a user.

Other embodiments relates to an identification and authentication system for authorized users of an infusion pump. The system including a touchless user authentication module, a touchless patient authentication module and a touchless infusion authentication module.

Further embodiments relate to a touchless programming and verification system for an infusion pump including one or more intermediary touchless devices. The system including an infusion pump providing safe and reliable touchless control and a touchless intermediary device for operation.

Further embodiments relate to an infusion pump having a touchless alarm silencing device. The infusion pump can include a proximity sensor serving as a touchless multifunction switch.

### BRIEF DESCRIPTION OF THE DRAWINGS

The invention can be more completely understood in consideration of the following detailed description of various embodiments of the invention in connection with the accompanying drawings, in which:

FIG. 1A is an example of a perspective view of a syringe type infusion pump equipped with a touchless user interface, according to an embodiment.

FIG. 1B is an example of a front view of an ambulatory type infusion pump equipped with a touchless user interface, according to an embodiment.

FIG. 2 is a block diagram of various elements of an infusion pump system equipped with a touchless user interface, according to an embodiment.

FIGS. 3A-D show representative examples of gesture-based touchless programming for an infusion pump, gesture-based or voice-based confirmation of programming, and a corresponding flowchart of a touchless programming and confirmation system, according to an embodiment.

FIGS. 4A-C show representative examples of voice-based touchless programming for an infusion pump, voice-based confirmation of programming, and a corresponding flowchart of a touchless programming and confirmation system, according to an embodiment.

FIGS. 5A-C show representative examples of voice-based touchless programming for an infusion pump, gesture-based confirmation of programming, and a corresponding flowchart of a touchless programming and confirmation system, according to an embodiment.

FIGS. 6A-C show representative examples of facial recognition-based touchless programming for an infusion pump, voice-based confirmation of programming, and a corresponding flowchart of a touchless programming and confirmation system, according to an embodiment.

FIGS. 7A-C show representative examples of voice-based touchless patient PCA dose requests for an infusion pump, confirmation of voice-based touchless patient PCA dose requests, and a corresponding flowchart of a touchless programming and confirmation system, according to an embodiment.

FIG. 8 is a flowchart of a touchless programming and verification system for an infusion pump utilizing a plurality of different touchless user interfaces, according to an embodiment.

FIG. 9 is a flowchart of a touchless programming and verification system for an infusion pump utilizing multiple different types of input to a touchless user interface, according to an embodiment.

FIG. 10 is a flowchart of a touchless programming and verification system for an infusion pump that includes biometric authentication of the user and patient identity, according to an embodiment.

FIG. 11 is a block diagram of various elements of a touchless programming and verification system for an infusion pump incorporating an intermediary touchless device, according to an embodiment.

FIG. 12 is a perspective view of an infusion pump equipped with proximity sensors, according to an embodiment.

FIG. 13 is a block diagram of various elements of an infusion pump system including one or more proximity sensors, according to an embodiment.

FIG. 14 is a block diagram of various elements of an infusion pump system having an authentication system, according to an embodiment.

FIG. 15 is a representative example of a display screen of a user interface in a user authentication system, according to an embodiment.

FIGS. 16A-E show representative examples of display screens of a user interface in an authentication system, according to an embodiment.

FIGS. 17 is a flowchart of a method relating to a touchless programming and verification system for an infusion pump including user authentication and pump programming authentication, according to an embodiment.

FIG. 18 is a flowchart of a method relating to a touchless programming and verification system for an infusion pump including user authentication and pump programming authentication, according to an embodiment.

The various embodiments can be embodied in other specific forms without departing from the essential attributes thereof; therefore, the illustrated embodiments should be considered in all respects as illustrative and not restrictive.

### DETAILED DESCRIPTION OF THE PREFERRED EMBODIMENTS

A number of areas in which improvements of infusion pumps and their designs are desired within hospital and medical environments have been recognized by applicants. One such area relates to safe and accurate infusion pump programming and operation. Accordingly, embodiments of this disclosure describe a multi-step technique of programming and confirmation requiring multiple different technologies or types of user inputs, that can help ensure errors in programming are avoided.

Applicants further recognize opportunities with respect to ensuring hygiene and cleanliness of infusion pumps themselves in medical environments. In general, hospitals and medical care facilities are intended to be clean. However, medical equipment and devices that are located in these areas are often touched by individuals who do not have clean hands and who could be a source of bacteria or other contamination to equipment surfaces. Due to the touchless design of the infusion pumps discussed in this disclosure, cleanliness potential is greatly enhanced when touchless user interface infusion pump systems are used. Specifically, the infusion pump touchless user interface system provides one less environment for touching and contamination during programming or reprogramming of the pump. The touchless user interface infusion pump system thereby becomes an unlikely place for harboring bacteria. Likewise, the speed at which doctors and nurses are able to act is improved as there is no need for ungloving and regloving when interacting with the infusion pump. The longevity of the device is improved as no physical programming interface is present for prolonged or harmful physical contact and manipulation of device components. Further, the lack of physical buttons allows for a maximum screen size, which can provide improved readability.

It has been found that problems have arisen at times in the past with respect to medical practitioners easily accessing the programming controls for an infusion pump. Typically, at least some of the pump and programming of its delivery mode and parameters are physically set up or adjusted locally by a medical practitioner, even when the pump is largely run by an on-board operating system contained within the pump. In some cases, accessing the infusion pump controls or user interface has been difficult due to a plurality of other medical devices and equipment physically surrounding a patient in need of a plurality of different medical treatments. Accordingly, the touchless user interface infusion pump system and its touchless user interfaces described in this disclosure help to solve or alleviate this problem as programming can occur at a greater distance from the device and does not require physical access directly adjacent the pump.

Advances in motion and voice recognition technologies have made new devices and innovations possible to correspondingly improve medical care facilities and the infusion pump field as described throughout this application. The present disclosure describes new designs, concepts, and ways to implement these touchless technologies within medical infusion pumps.

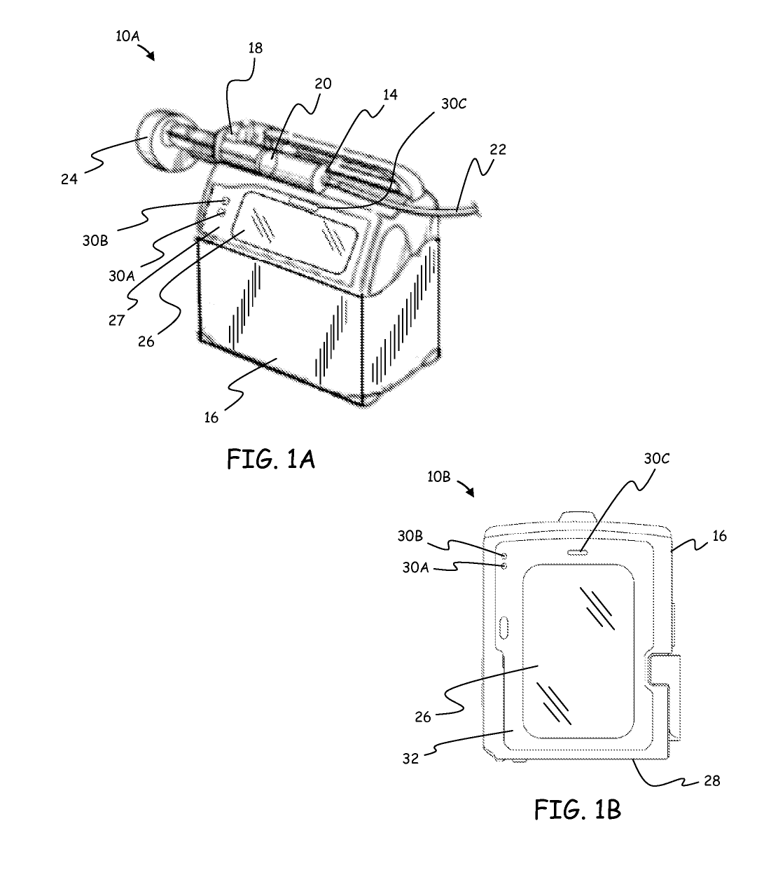

FIGS. 1A and 1B show examples of infusion pumps 10A and 10B, respectively, (also referred to more generally in this disclosure by numeral 10) implementing touchless user interfaces. In general, infusion pump 10A is a syringe-type pump that can be used to deliver a wide range of drug therapies and treatments. The infusion pump 10A includes a pharmaceutical container, or syringe 14, which is supported on and secured by housing 16 and clamp 18, respectively. The syringe 14 includes a plunger 20 that forces fluid outwardly from syringe 14 via infusion line 22 that is connected to a patient. A motor and lead screw arrangement internal to the housing 16 cooperatively actuates a pusher, or plunger driver mechanism 24, to move the plunger 20. A sensor, which is typically internal to the plunger driver mechanism 24, monitors fluid force as desired per system specifications.

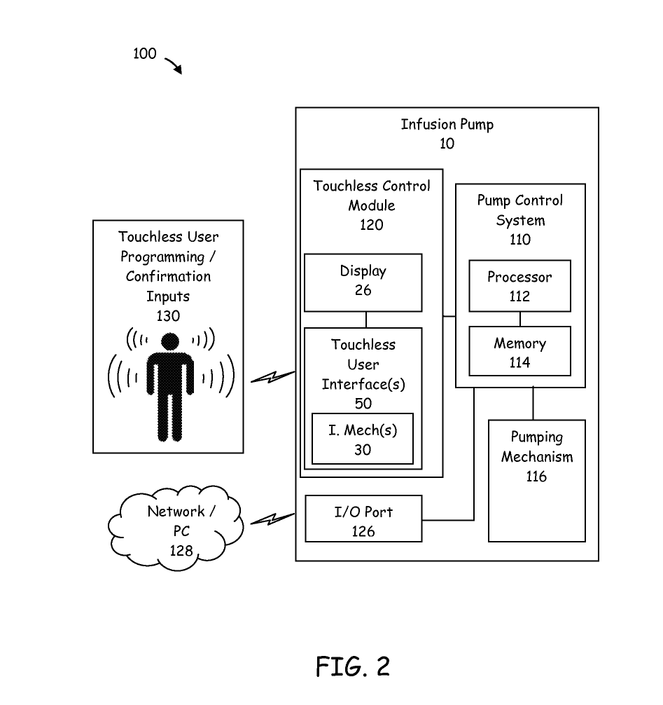

The pump housing 16 includes a touchless display 26 on the front face 27 of the infusion pump 10A. The touchless display 26 is prominently featured and viewable from a significant distance such that users can review the screen even when they are not directly adjacent or in close proximity to infusion pump 10A. Other locations for the touchless display 26 on or around the infusion pump 10A are contemplated as well. Further, at least one touchless operator input mechanism 30 (or individually as 30A, 30B, 30C, . . . etc.) is present in the device as well. These touchless operator input mechanisms 30, are cameras or sensors which enable the one or more touchless user interfaces 50 of the pump 10A to function as depicted in FIG. 2. The touchless user interface(s) 50 can utilize one or more types of touchless sensing technology and can be gesture-based or voice-based, for example. Other touchless sensing technologies can incorporate facial recognition, optical character recognition or label recognition, for example.

Referring to FIGS. 1A, 1B, and 2, the touchless operator input mechanisms 30 are generally subparts of one or more touchless user interface(s) 50. A touchless user interface 50 can rely on gesture recognition devices utilizing one or more touchless operator input mechanisms 30 in the form of cameras, electric field sensors, surface or projected capacitance sensors, or other touchless gesture recognition technology. Although depicted separately in FIG. 2, in some embodiments, the touchless display(s) 26 can be considered a part of the touchless user interface 50 as well. In some embodiments, multiple touchless user interfaces 50 will share a single display 26 and in others, separate displays 26 for each touchless user interface 50 will be used.

Referring to FIGS. 1A, 1B, and 2, camera-based, gesture-based recognition devices use cameras to recognize body, arm, hand, head or finger motion. In some embodiments, the infusion pump 10A implements a motion sensing technology similar to that found in devices like Microsoft's Kinect™ or Leap Motion's peripheral device controller. Specifically, Kinect™ uses software technology and range camera technology to provide a system capable of interpreting specific human gestures to make hands-free control of electronic devices possible. It does this using an infrared projector and camera and microchip to track the movement of objects and individuals in three dimensions. Alternatively, the current Leap Motion peripheral device controller is a small USB peripheral device that can be placed on a flat surface near the device being controlled, for example. Using a plurality of cameras and a plurality of infrared LEDs, the Leap Motion device observes a roughly hemispherical area to a distance of a few feet. It is designed to track fingers, or similar items such as a pen, which cross into the observed area. Other types of camera-based, gesture-based recognition devices are contemplated as well.

Accordingly, using these types of camera-based, gesture-based recognition technologies in an infusion pump enables a medical practitioner to adjust programming of the infusion pump 10A with a gestured swipe, pinch, or press within close proximity to the screen rather than a button push. Touchless operator input mechanisms 30 can represent cameras, LEDs or other components that might be necessary parts of a camera-based, gesture-based, touchless user interface. The locations, sizes, shapes, or number of these mechanisms 30 shown in the figures are merely illustrative and are not intended to be limiting or restrictive to any particular design.

Electric field sensor recognition devices can include gesture-based recognition of body or finger motion. Accordingly, the infusion pump 10A can alternatively implement an electric field sensor in certain embodiments. An electric field sensor can include technology like Microchip GestIC 3D sensor technology that utilizes an electric field for advanced proximity sensing. It allows user interface applications by detection, tracking and classification of user hand or finger motion in free-space. In general, a quasi-static electrical near field is created that can sense conductive objects like the human body which distort the electric field distribution when they intrude the sensing area. Accordingly, using these types of electric field-based gesture-based motion sensing technologies in an infusion pump allows a medical practitioner to adjust programming of the pump with a gestured swipe, pinch, or press within close proximity to the screen rather than a button push. Accordingly, touchless operator input mechanisms 30 can represent field sensors or other components that would be necessary parts of an electric field sensor-based, gesture-based, touchless user interface 50. The locations, sizes, shapes, or number of mechanisms 30 shown in the figures are merely illustrative and are not intended to be limiting or restrictive to any particular design.

Surface or projected capacitance sensors can include gesture recognition of body or finger motion. A surface or projected capacitance sensor can alternatively be implemented in one or more touchless user interfaces 50 in the infusion pump 10A as well. Such a sensor can include technology like Azoteq ProxSense technology, which measure the capacitance on an electrode with high sensitivity circuits. Using this capacitance information, objects like the human body can be sensed when they intrude the sensing area. Accordingly, using these types of surface or projected capacitance-based, gesture-based, motion sensing technologies in an infusion pump 10A allows a medical practitioner to adjust programming of the pump with a gestured swipe, pinch, or press within close proximity to the screen rather than a button push. Accordingly, touchless operator input mechanisms 30 can represent capacitance sensors or other components that would be necessary parts of capacitance sensor-based, gesture-based, touchless user interface. The locations, sizes, shapes, or number of mechanisms 30 shown in the figures are merely illustrative and are not intended to be limiting or restrictive to any particular design.

Infusion pump 10B shown in FIG. 1B is an example of an ambulatory pump that can be used to deliver a wide range of drug therapies and treatments. Such ambulatory pumps 10B can be worn by a user for in-home care, but are also often provided in ambulatory, pole-mounted arrangements within hospitals and other medical care facilities. The infusion pump 10B generally includes a peristaltic type infusion pump mechanism which controls the flow of medication from a reservoir of fluid through a conduit passing along the bottom surface 28 of the pump 10B. This fluid can be from a cassette reservoir (not shown) that is attached to the bottom of the pump 10B at surface 28, or from an IV bag or other fluid source (not shown) that is similarly connected to pump 10B via an adapter plate at surface 28. Specifically, the pump 10B uses valves and an expulsor located on the bottom of the pump 10 to selectively squeeze a tube of fluid to effect the movement of fluid through the tube and to a patient in peristaltic pumping fashion.

The infusion pump 10B has a pump housing 16 including a touchless display 26 located on the face 32 of the pump. Similar to infusion pump 10A, the touchless display 26 of infusion pump 10B is prominently featured and readily viewable. At least one touchless operator interface input mechanism 30 (or alternatively 30A, 30B, 30C, . . . etc.) is present in the device as well. The touchless operator interface mechanisms 30 can be cameras, electric field sensors, or surface or projected capacitance sensors, for example, that would be necessary components of a gesture-based, touchless user interfaces 50. Touchless operator interface mechanisms 30 can be voice or sound sensors in a voice-based touchless user interface 50. With respect to both figures 1A and 1B, the types of gesture-based touchless user interfaces 50 include, but are not limited to those discussed above. The locations, sizes, shapes, or number of input mechanisms 30 shown in the figures are merely illustrative and are not intended to be limiting or restrictive to any particular design. Operator interface input mechanisms 30 can be largely contained within the pump housing 16 or can be external and operate as peripheral devices to the infusion pumps 10A and 10B.

FIG. 2 is a block diagram of various elements of a touchless infusion pump system 100. The system 100 includes an infusion pump 10 having a pump control system 110 with a processor 112 and memory 114 programmable with selected protocols, profiles and other settings for controlling operation of a pumping mechanism 116 such as, e.g., the aforementioned syringe and peristaltic type mechanisms. The infusion pump 10 also includes a touchless control module 120 for relaying commands to the pump control system 110. Touchless control module 120 includes at least one touchless user interface 50 utilizing a touchless operator input technology including input mechanism(s) 30, that works cohesively with a display screen 26. (In cases where a plurality of touchless user interfaces 50 are present, the user interfaces can alternatively be referred to in this disclosure by an alpha-numeric nomenclature, such as 50A, 50B, 50C, . . . etc. together with their corresponding touchless user input mechanism(s) 30A, 30B, 30C, . . . as well). In some cases the display 26 will be considered part of the touchless user interface(s) 50.

The infusion pump 10 includes a USB port or other appropriate input/output (I/O) interface port 126 for connecting the infusion pump 10 to a network or computer 128 having software designed to interface with the infusion pump 10. Power to the infusion pump 10 is accomplished via an AC power cord or internally provided battery.

Touchless user inputs 130 to the system are provided by touchless programming of a user such as a nurse, physician, or other medical practitioner. These inputs 130 can include: gestures; voice commands; facial movements or expressions; finger, hand, head, body and arm movements; or other inputs that do not require physical contact with the infusion pump 10. The inputs 130 are generally communicated, sensed or received by the touchless operator input mechanisms 30 of a touchless user interface 50. As previously mentioned, such touchless operator input mechanisms 30 can include cameras or sensors of electric field, capacitance, or sound, for example.

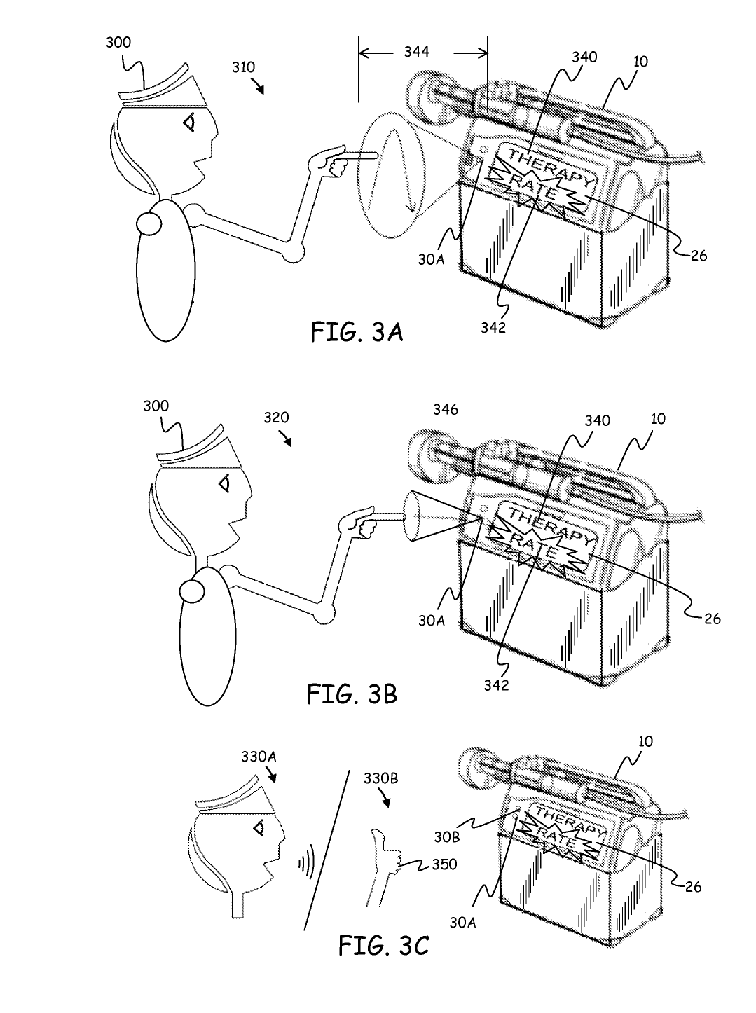

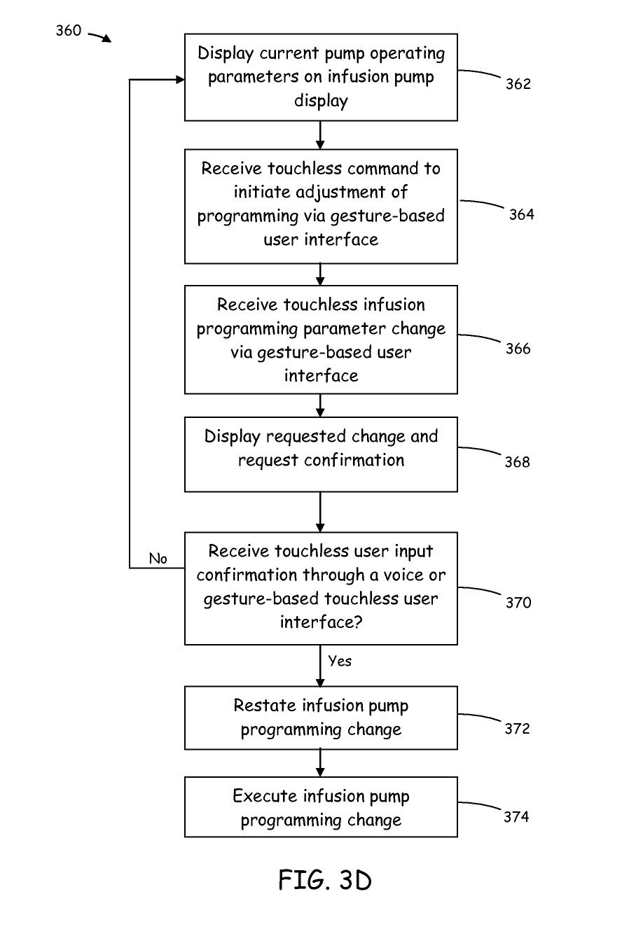

FIGS. 3A and 3B show representative examples of gesture-based touchless programming commands 310 and 320 for an infusion pump 10 by a user 300. FIG. 3C shows a representative example of touchless gesture-based confirmation 330A or voice-based confirmation 330B of programming for an infusion pump 10. An example of a possible programming and confirmation interaction can include steps similar to those discussed in the following paragraphs.

First, in FIG. 3A a nurse or other authorized user 300 views the displayed therapy 340 being administered and rate 342 of administration on the display 26. Next, the user 300 initiates a programming change to the input mechanism 30A of the gesture-based user interface 50A. This can occur by a user 300 making a gesture of forming a letter “A” with his/her finger in front of the pump 10 indicating a request to adjust the currently displayed rate of infusion 342 on the display 26, for example. The gesture-based touchless user interface 50A can use cameras as the input mechanism 30A, but can use an alternate type of gesture-based touchless user interface 50A and input mechanism 30A as well. In some cases, the user's gestures may need to be within a certain distance 344 of such a camera (such as within twelve inches, for example). Next, the infusion pump 10 flashes an inversion of the screen (two times per second, for example) to show that it is being modified.

As depicted in FIG. 3B, the user 300 can next adjust the rate of infusion 342 by performing a flicking motion to the right or to the left with his/her index finger directly in front of an appropriate area of the display 26 to increase the infusion rate 342 displayed. A 0.1 mL/hr decrease or increase in rate 342 per left or right flick, respectively, will accordingly be displayed on the infusion pump display 26, for example. For an increase of, for example, 0.7 mL/hr, seven right flick gestures can be required. In some cases, this flicking motion will need to take place within a certain distance 346 from the display screen 26 (such as one to two inches, for example). When the user 300 is finished adjusting the infusion parameter, another gesture, such as a single up and down hand motion can be made (not shown) and/or required to communicate that adjustment is complete.

Next, as shown in FIG. 3C, a confirmation 330A or 330B is performed. In the case of using confirmation 330B, the pump first displays a confirmation message on the display 26 on the front of the infusion pump 10, such as “Confirm 0.7 mL/hr increase with verification motion”. The user 300 can then perform a unique gesture 350 reserved for authenticating changes as at 330B. This gesture 350 can be thumbs up motion or a user-specific motion signature. The infusion pump 10 responds by displaying “Adjustment Accepted” temporarily (for one to two seconds) and then increasing the dosage as specified by the programming change.

Alternatively, a confirmation 330A can be used which involves a second touchless user interface 50B using a separate touchless technology and separate operator input mechanism 30B. In one example, the pump 10 can display a confirmation message on the display 26 that states “Confirm 0.7 mL/hr increase by stating increase”. The user 300 would then respond by verbally stating for example “0.7 mL/hr increase confirmed” or “Yes, increase”. The infusion pump 10 then uses a second touchless user interface 50B contained in the pump to apply voice recognition to verify the infusion rate increase that was verbally stated by the user 300. The infusion pump 10 then responds by displaying “Adjustment Accepted” temporarily (for one to two seconds) and increasing the dosage as specified by the programming change.

There are advantages to using multiple forms of touchless user interface as there is a reduced potential for unwanted entry errors to be carried out when the commands and confirmations must be consistent across multiple types of touchless human interactions and communications. Accordingly, this arrangement greatly enhances a potential of maintaining the “five rights” and expectations of safety and accuracy in infusion pump programming. Specifically, this expectation is satisfied, even when using a touchless type of user interface that is newly introduced to users.

At times, however, use of the same type of touchless user interface 50 for programming and confirmation has the advantage of only requiring the components and cost of a single touchless interface 50 on board the infusion pump 10. Accordingly, some embodiments can utilize the same touchless user interface 50, but use two distinct input types within that touchless user interface 50 to provide enhanced safety and programming assurances.

FIG. 3D provides a general flowchart 360 of a touchless programming and confirmation system similar to the one described in FIGS. 3A-C. First, at 362 the touchless infusion pump display 26 displays one or more pump operating parameters or other items of pump information. These operating parameters or pump information can include the type of therapy 340 and the rate of infusion 342, but can also include infusion delivery profile information of any of a multitude of various items of information related to pump operation and status, such as rate, medication type, time, or route of delivery. Next, at 364 the infusion pump receives a touchless command to initiate adjustment of programming via the gesture-based user interface 50A. Next, at 366 the infusion pump 10 receives a touchless infusion programming change via the gesture based user interface 50A. At 368, the infusion pump 10 presents the requested change on the display screen 26 and requests user confirmation. At 370, a determination is made based on whether the infusion pump 10 received a touchless user input confirmation through a voice-based user interface 50B or gesture-based touchless user interface 50A. If confirmation is received, the infusion pump 10 proceeds to restate the infusion pump programming change at 372. If no confirmation is received, the steps 360 are repeated from the beginning at 362. If 372 is reached, next the infusion pump 10 proceeds to execute the infusion pump programming change at 374.

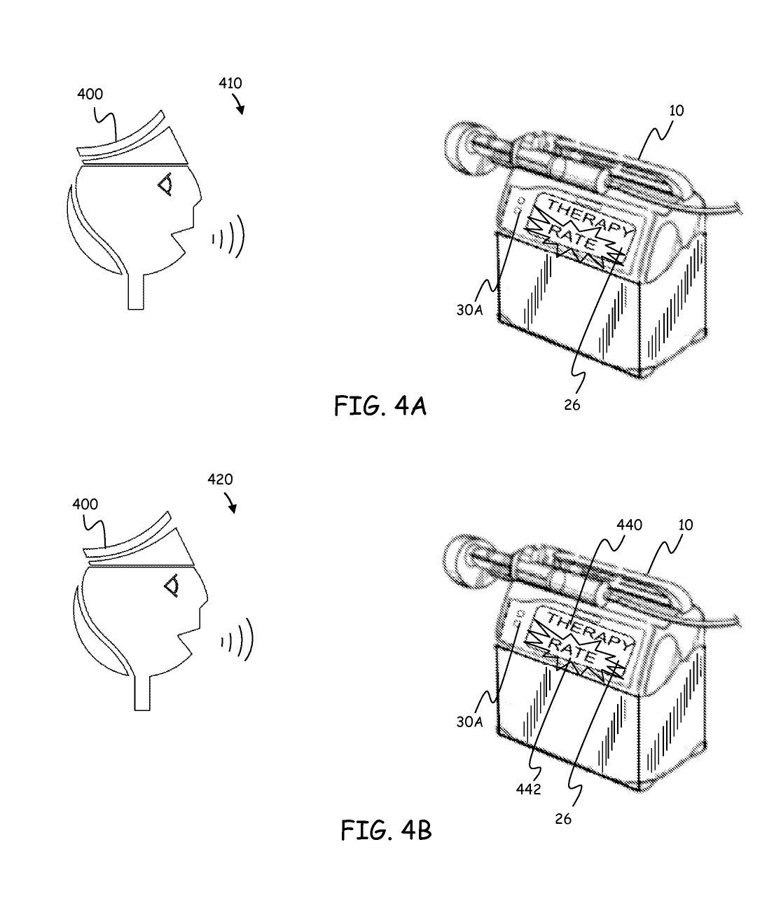

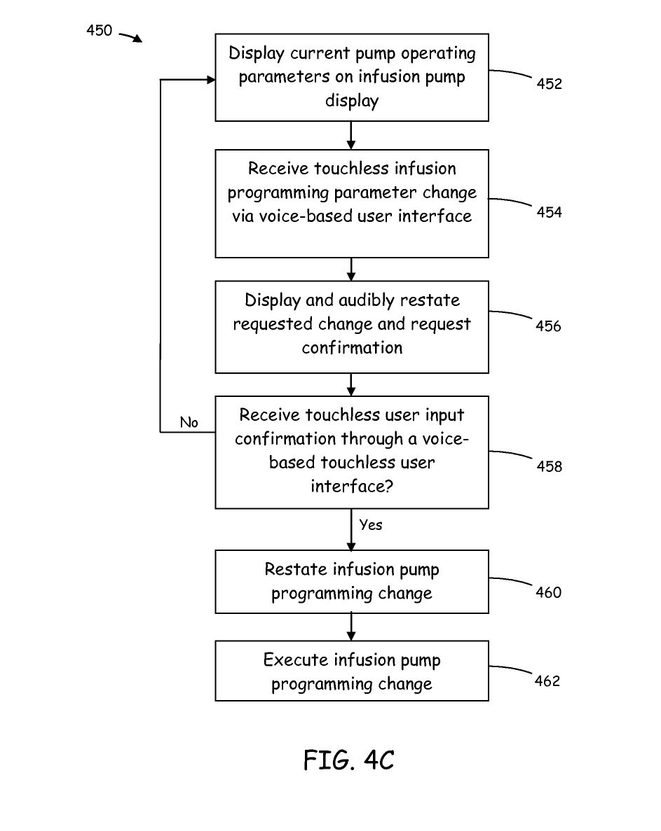

FIGS. 4A and 4B show representative examples of voice-based touchless programming and confirmation of programming for an infusion pump, respectively. First, in FIG. 4A a nurse or other user 400 initiates a programming command 410 by speaking a programming parameter change to the operator input mechanism 30A of a voice-based touchless user interface 50A contained within infusion pump 10. In this instance, the operator input mechanism 30A is a voice or speech sensor and software for recognizing voice inputs and commands. The user 400 might state, for example, “Pump, Dobutamine, increase 5 mL/hr”. The infusion pump 10 can then respond verbally with a statement, such as “Dobutamine to increase 5 mL/hr? State password to confirm”. At the same time, “Dobutamine” would be presented on the display 26 of the infusion pump 10 with a flashing inversion (two times per second, for example). Next, as shown in FIG. 4B, the user 400 audibly speaks a user specific password or makes a specific sound to the voice-based touchless user interface 50A as a confirmation action 420 to confirm the programming change. The infusion pump 10 responds with a verbal confirmation of the infusion pump 10 programming change stating, for example, “Confirmation recognized, increasing Dobutamine 5 mL per hour” followed by an implementation of the programming change.

FIG. 4C provides a general flowchart 450 of a touchless programming and confirmation system analogous to the ones described in FIGS. 4A-B. First, at 452 the touchless infusion pump display 26 displays one or more pump operating parameters or pump information. This type of information can include the type of therapy 440 and the rate of infusion 442, but can also include any of a multitude of various items of information including delivery profile information or any other type of graphical information regarding infusion that can be displayed. Next, at 454 the infusion pump receives a voice command from a user to initiate adjustment of programming via the voice sensors 30A of the voice-based user interface 50A. At 456, the infusion pump 10 presents the requested change on the display screen 26 and audibly requests user confirmation. At 458, a determination is made based on whether the infusion pump 10 received a touchless user input confirmation through a voice-based user interface 50A. If confirmation is received, the infusion pump 10 proceeds to restate the infusion pump programming change at 460. If no confirmation is received, the steps 450 are repeated from the beginning at 452. If 460 is reached, next the infusion pump 10 proceeds to execute the infusion pump programming change at 462.

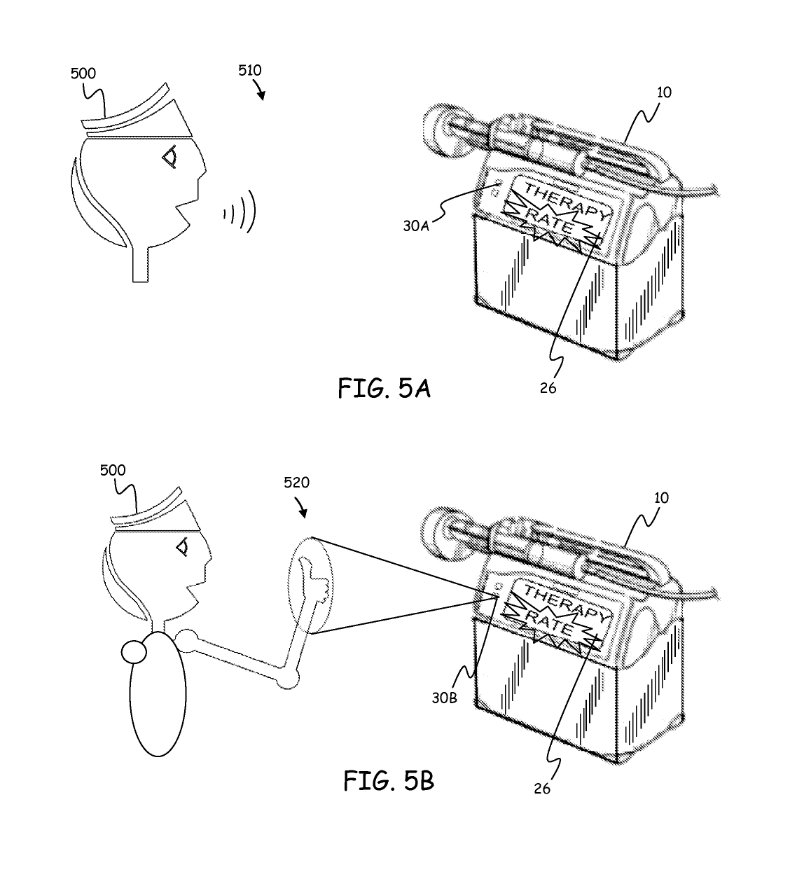

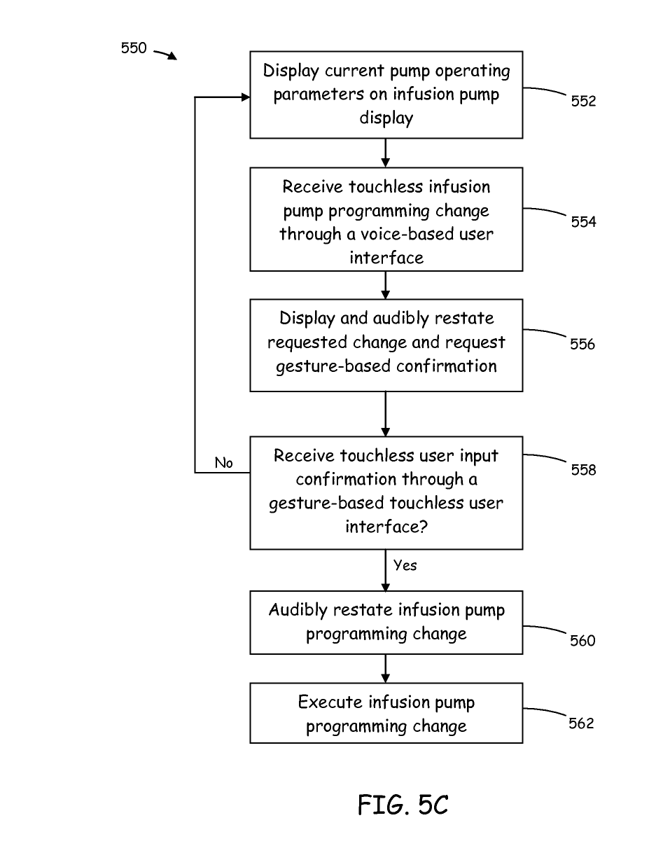

FIGS. 5A and 5B show an embodiment depicting representative examples of voice-based touchless programming and gesture-based confirmation of programming for an infusion pump. First, in FIG. 5A a nurse or other user 500 initiates a programming action 510 to a touchless user interface 50A that allows for voice or speech recognition via the operator input mechanism 30A. In this instance, the operator input mechanism 30A is a voice or speech sensor and software for recognizing voice inputs and voice-based commands. The user 500 might state, for example, “Pump, Dobutamine, increase 5 mL/hr”, as a voice command. The infusion pump 10 would respond verbally with “Dobutamine to increase 5 mL/hr? Thumbs up to confirm”. At the same time, “Dobutamine” is presented on the display 26 of the infusion pump 10 with a flashing inversion (two times per second, for example). Next, as shown in FIG. 5B, the user 500 would provide a thumbs up confirmation motion 520 in front of the touchless interface 30B (within twelve inches, for example). This is followed by a verbal confirmation of the infusion pump 10 stating “Confirmation recognized, increasing Dobutamine 5 mL per hour”.

FIG. 5C is a general flowchart 550 of a touchless programming and confirmation system analogous to the one described in FIGS. 5A-B. First, at 552 the touchless infusion pump display 26 displays one or more pump operating parameters. Next, at 554 the infusion pump 10 receives a touchless command to initiate adjustment of pump programming via the voice-based user interface 50A. Next, at 556 the infusion pump 10 displays and audibly restates the requested programming change and requests gesture-based confirmation from the user 500. Next at 558, if no confirmation is received, the display 26 reverts back to display of current pump parameters as at 552. If, however, at 558 confirmation of the touchless user input is received through a gesture-based touchless user interface, then the pump audibly restates the infusion pump programming change at 560 and then proceeds to execute the requested infusion pump programming change at 562.

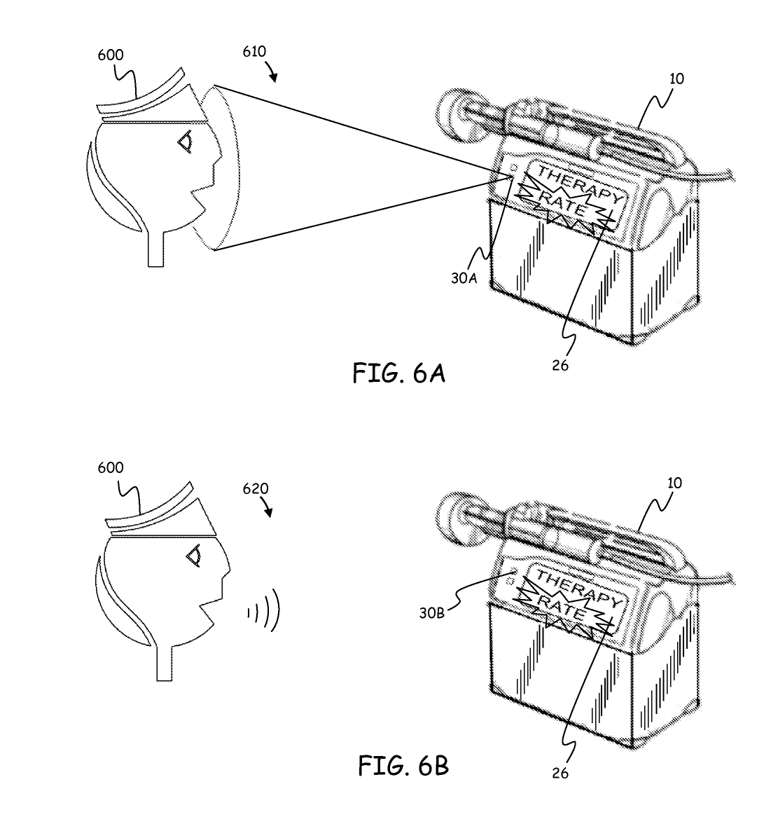

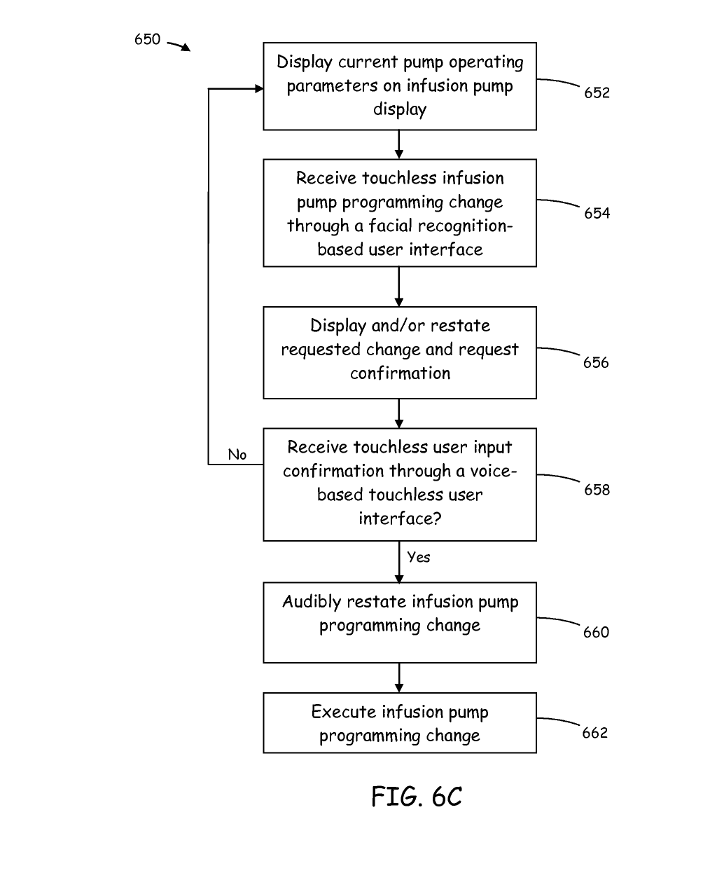

FIGS. 6A and 6B show representative examples of facial recognition-based touchless programming and voice-based confirmation of programming for an infusion pump. In FIG. 6A, a touchless user interface is used to recognize facial features and movements of user 600 in order to carry out infusion pump programming commands. Various facial recognition devices and camera-based technologies can be used to accomplish this. Both facial expressions and facial movements can provide programming commands 610. Confirmation of these commands can be done with a variety of touchless interfaces 50. In FIG. 6B, an example of a user 600 utilizing voice-based confirmation 620 of a pump parameter change is shown.

FIG. 6C is a general flowchart 650 of a touchless programming and confirmation system similar to the one described in FIGS. 6A-B. First, at 652 the touchless infusion pump display 26 displays one or more pump operating parameters. Next, at 654 the infusion pump 10 receives a touchless command to initiate adjustment of pump programming via the facial recognition-based user interface 50A. Next, at 656 the pump displays and/or audibly restates the requested programming change and requests voice-based confirmation from the user 600. Next at 658, if no confirmation is received, the display 26 reverts back to display of current pump parameters as at 652. If, however, at 658 confirmation of the touchless user input is received through a voice-based touchless user interface, then the infusion pump 10 audibly restates the infusion pump programming change at 660 and then proceeds to execute the requested infusion pump programming change at 662.

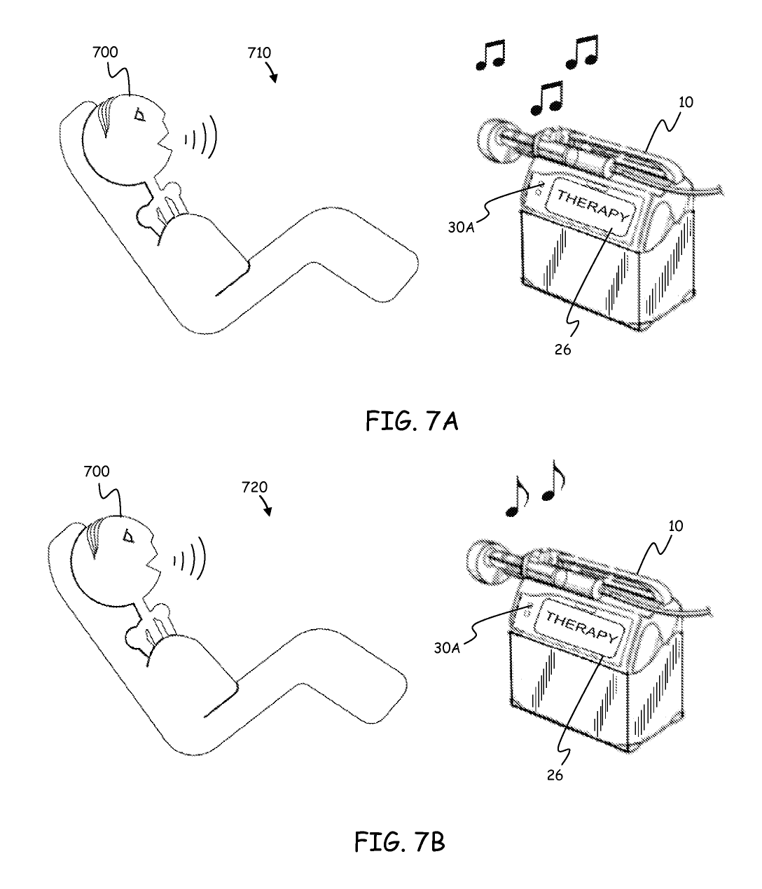

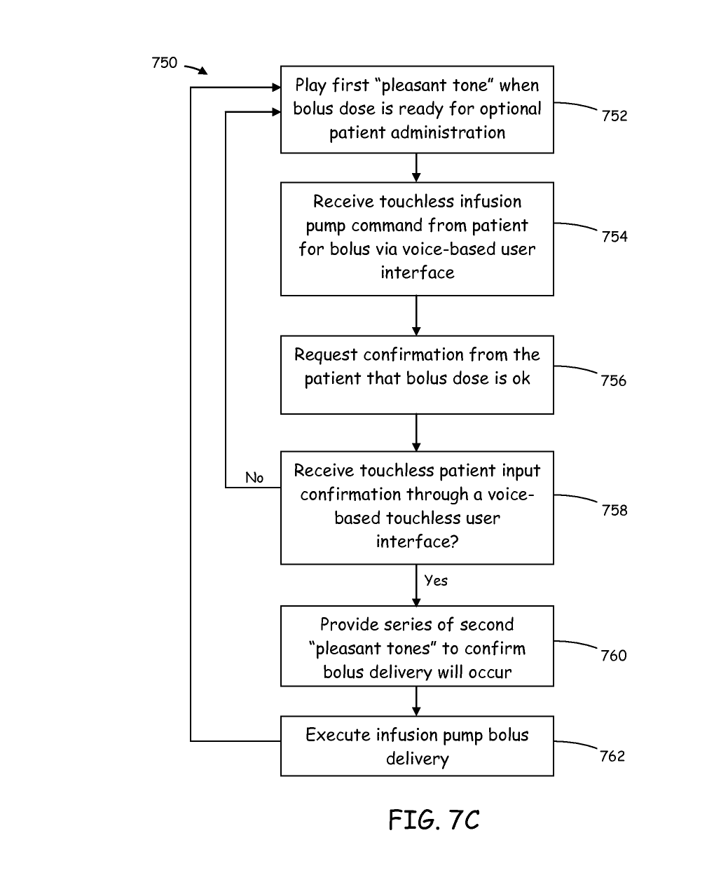

The embodiments disclosed in FIGS. 7A and 7B show representative examples of voice-based touchless PCA dose requests and confirmation of voice-based touchless PCA dose requests for an infusion pump 10, respectively.

In FIG. 7A, an infusion pump indicates that a bolus dose of medication is available for delivery to patient 700 upon request via the display 26 and a broadcast of a first set of audible pleasant tones. Patient 700 is shown giving a voice programming command 710 to the touchless user interface 50A via input mechanism 30A of the infusion pump 10 requesting a bolus dose of pain medication. For example, the patient 700 can initiate a bolus of pain medication by stating “pain reliever, please”. The infusion pump 10 recognizes this command and replies with an audible response for confirmation, stating “Dose OK?”. Next, in FIG. 7B the patient responds by stating “OK” as a confirmation command 720. Confirmation is then followed by broadcast of a second set of pleasant tones, different from the first set, by the infusion pump 10 indicating that a dose is being administered. When the patient 700 is eligible for the next bolus dose, the first set of pleasant tones is played to indicate that a dose is ready. This would restart the previous sequence of bolus and confirmation commands between the patient 700 and pump 10.

Allowing patient control of administration of pain medication via a PCA delivery mode has proved to be particularly advantageous in some types of infusions. In existing infusion pumps, patient requests for medication are typically delivered via a manually operated remote control that is connected to the infusion pump by a cord. In the example of FIGS. 7A and 7B, there is no cord, but rather, the patient gives commands to the infusion pump 10 verbally. This is especially advantageous as there is no connector present for water ingress, no dose cord to lose or break, no possibility of incorrectly coupling the connector to the pump, and no EMC access. Further, such a system requires less costs for connector and electrical components, no break in the sterile field if used by a nurse, and no chance of confusing it with the nurse call button. In some embodiments, an audio record can be kept by the infusion pump for dosing verification and data collection (˜5 seconds, for example).

FIG. 7C is a general flowchart 750 of a touchless programming and confirmation system similar to the one described in FIGS. 7A-B. First, at 752 the touchless infusion pump plays a first set of pleasant tones to indicate that a bolus dose of medication is ready for optional patient administration upon request. Next, at 754 the infusion pump 10 receives a touchless command from the patient 700 to provide a bolus dose of medication via the voice-based user interface 50A. Next, at 756 the pump requests voice-based confirmation from the patient 700 that a bolus dose is desired. Next at 758, if no confirmation is received, the infusion pump reverts back to broadcast of a first set of pleasant tones indicating that a bolus dose of medication is available upon request as at 752. If, however, at 758 confirmation of the touchless user input is received through a voice-based touchless user interface, then the infusion pump 10 audibly provides a second set of pleasant tones which confirm that bolus delivery is about to occur at 760. The infusion pump 10 then proceeds to execute the medication bolus delivery at 762.

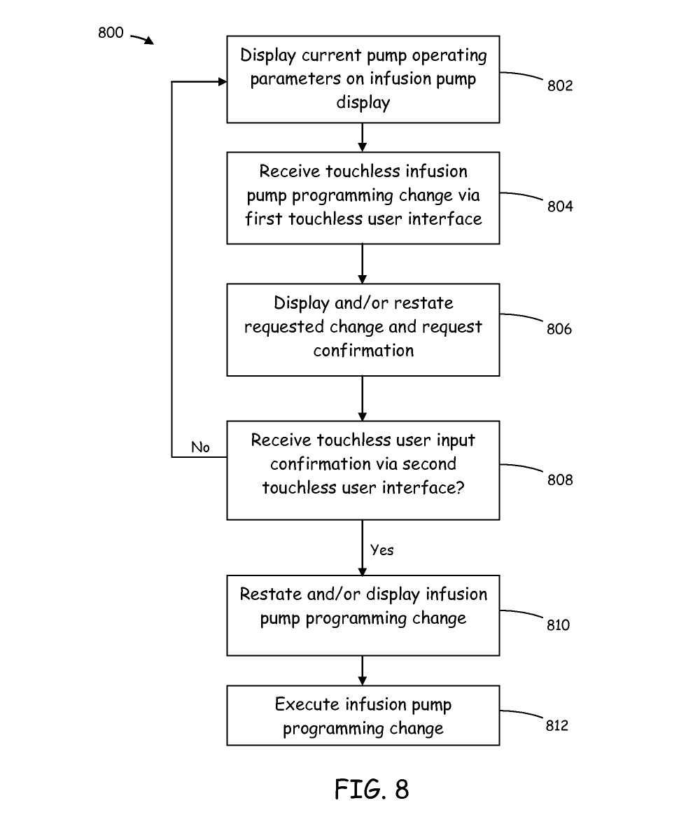

FIG. 8 is a flowchart of a touchless programming and verification system method 800 for an infusion pump 10 utilizing multiple touchless user interfaces. First, at 802, the pump displays at least one operating parameter of the infusion pump. Next, at 804, the infusion pump receives a touchless infusion pump programming change via a first touchless user interface 50A, present in the infusion pump 10. Next, at 806 the infusion pump displays and/or restates the requested change and requests confirmation from the user. Next, at 808 if no touchless user input of confirmation is received via a second touchless user interface of the infusion pump 10, then the pump reverts back to the initial pump display containing current pump operating parameters such that a new programming change can be newly requested. If, however, at 808 a touchless user input of confirmation is received via the second touchless user interface 50B of the infusion pump 10, then at 810 the infusion pump 10 will restate and/or display the requested infusion pump programming changes. Finally, at 812 the infusion pump will execute the infusion pump programming changes.

FIG. 9 is a flowchart of a touchless programming and verification system method 820 for an infusion pump utilizing multiple types of touchless user input and/or techniques. First, at 822, the pump displays at least one current operating parameter of the infusion pump 10 on the display 26. Next, at 824, the infusion pump 10 receives a touchless infusion pump programming change via a first type of touchless input to a touchless user interface 50A, present in the infusion pump 10. Next, at 826 the infusion pump 10 displays and/or restates the requested change and requests confirmation from the user. Next, at 828 if no touchless user input is received via a second type of touchless input to the touchless user interface 50A of the infusion pump 10, then the pump reverts back to the initial pump display containing current pump operating parameters such that a new programming change can be newly requested. If, however, at 828 a second type of touchless user input of confirmation is received via the second touchless user interface 50B of the infusion pump 10, then at 830 the infusion pump 10 will restate and/or display the requested infusion pump programming changes. Types of touchless inputs by the user, such as the first and second types of touchless input discussed may include voice-based instructions, gesture-based movements including body, arm hand head finger, facial recognition or movement, touchless fingerprint inputs, or positioning for proximity sensor(s) for example. Finally, at 832 the infusion pump will execute the infusion pump programming changes.

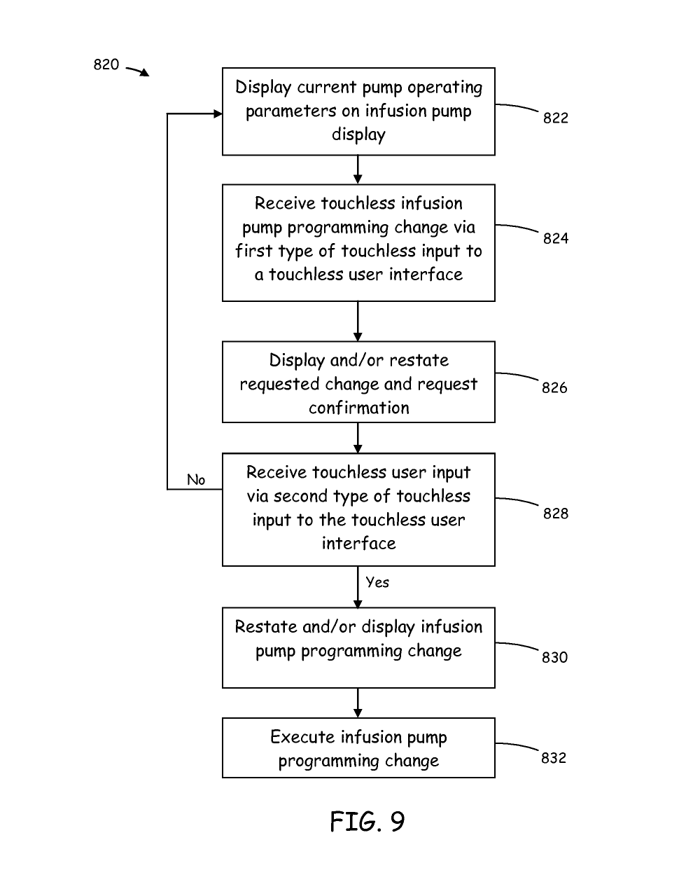

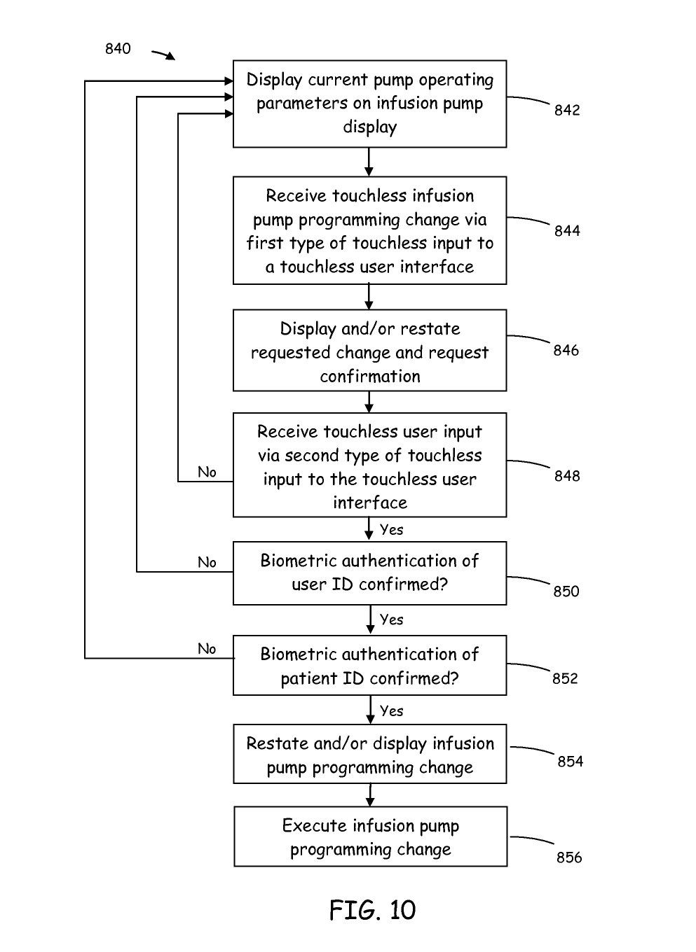

FIG. 10 is a flowchart of a touchless programming and verification system method 840 which further includes user or patient identity confirmation. In this method, the infusion pump 10 first displays, at 842, at least one operating parameter of the infusion pump 10. Next, at 844, the infusion pump receives a touchless infusion pump programming change via a first touchless user interface 50A, present in the infusion pump 10. Next, at 846 the infusion pump displays and/or restates the requested change and requests confirmation from the user. Next, at 848 if no touchless user input of confirmation is received via a second touchless user interface of the infusion pump 10, then the pump reverts back to the initial pump display containing current pump operating parameters such that a new programming change can be newly requested. If, however, at 848 a touchless user input of confirmation is received via the second touchless user interface 50B of the infusion pump 10, then at 850 the infusion pump 10 request a touchless form of biometric authentication of the user of the pump interface to continue. If confirmed, then at 852, the infusion pump 10 will request a touchless form of biometric authentication of the patient receiving treatment from the infusion pump 10. Next, if confirmed, then at 854 the infusion pump 10 will restate and/or display the requested infusion pump programming changes. Finally, at 856 the infusion pump will execute the infusion pump programming changes. In some embodiments, only biometric authentication of either the user ID or patient ID is required, and accordingly, one of 852 or 854 would not be required. In other embodiments, the biometric authentication of the user ID and patient ID may precede the receipt of programming changes.

Touchless forms of biometric authentication of the user or patient, as discussed in 850 and 852 can include one or more of the following: Retinal scanning, facial recognition, voice recognition, voice passcode, touchless fingerprint recognition, gestured user-specific passcode. The components required for one or more of these authentication methods can be implemented into an infusion pump 10. Other forms of touchless biometric authentication technologies can be possible as well.

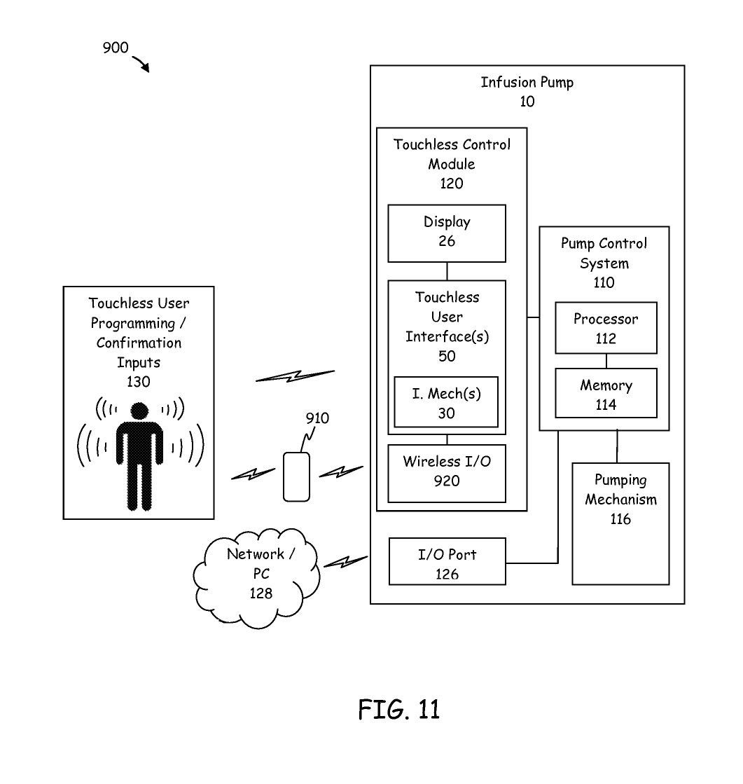

FIG. 11 is a block diagram of various elements of a touchless infusion pump system 900 incorporating an intermediary touchless device 910, according to an embodiment. In each of the embodiments disclosed, it can be desirable for the user of the touchless user interface to have a further intermediary device 910 available to him/her to supplement any interactions between the user and the infusion pump 10. Examples of such intermediary devices 910 can include smart phones, tablets, or other wireless communication devices. Accordingly, the touchless infusion pump system 900 diagram further depicts an intermediary device 910 as well as a wireless I/O device 920 within the infusion pump 10 for receiving supplemental inputs from the intermediary device 910. Accordingly, standard and additional steps of programming command and confirmation can be enhanced by the additional capabilities of such an intermediary wireless device.

Embodiments of the infusion pump with touchless user interface should be understood to include a variety of features and methods providing for touchless interactions with and convenient operation of infusions pumps. Embodiments enabling touchless commands should not be limited to programming of an infusion pump, per se, but also include simple tasks such as touchless silencing of an alarm, pausing an infusion, or starting an infusion. Such simplified commands can be implemented with touchless operator input mechanisms 30 as discussed above which may include proximity sensors in some embodiments or may be implemented with additional proximity sensors relying on infrared, ultrasonic, capacitive, inductive, optical, RFID, or other technologies.

The benefits of being able to perform these type of basic pump functions in a touchless matter can be readily realized in environments such as Intensive Care Units (ICU) as clinicians must wash their hands each time they touch a piece of equipment. This can result in significant delays in care, frustration of users, and enlisting further personnel to touch the pump at times. Accordingly, the capability to perform simple tasks is clearly a desirable feature of care professionals who interact with infusion pumps and like devices.

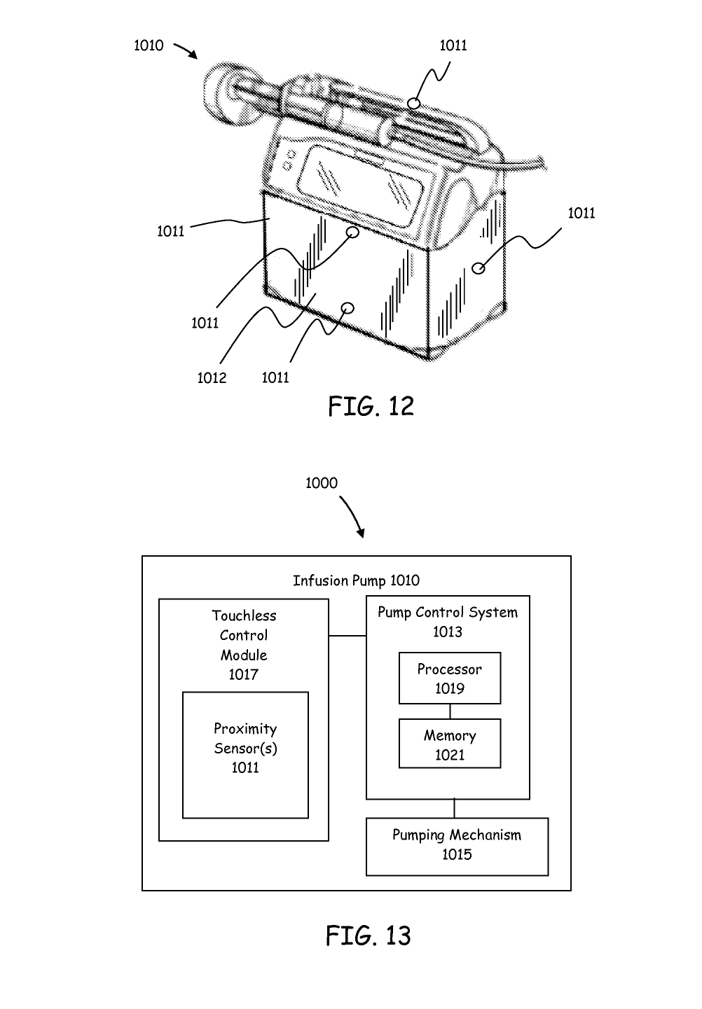

As shown in FIG. 12, an infusion pump 1010 can have one or more proximity sensors 1011 on or in housing 1012 in some embodiments. In some embodiments having only one proximity sensor, the proximity sensor 1011 can be utilized as a multifunction switch. The function of the switch can vary depending on the status of the pump. For example, if an alarm on the infusion pump 1010 is active due to an error or other safety alert, the switch can simply silence the alarm using a touchless interaction that provides a touchless command. Similarly, if an infusion pump 1010 is running an infusion, the switch can pause the pump 1010 using a touchless interaction that provides a touchless command. If the pump 1010 is paused, then the switch can start the infusion using a touchless interaction that provides a touchless command.

Additionally, the pump could utilize a series of non-contact interactions to provide incremental responses. For example, a first hand wave or gesture recognized by a sensor 1011 can be used as a command to silence an audible alarm, but leave a warning light flashing on the infusion pump 1010. A second hand wave or gesture recognized by a sensor 1011 within a prescribed period of time (such as 2 seconds, for example) can be used as a command to dismiss or extinguish the warning light. Alternatively, use of more sophisticated touchless sensors or touchless operator input mechanisms 30 providing partial or full touchless interaction with a pump display, using cameras or IR sensing for example, is possible as described earlier.

In the embodiment of FIG. 12, five proximity sensors 1011 are spread around the pump 1010 such that they can be used as a type of “five-way touchless joystick” for a higher level of interaction with the user interface. Sensors on the sides of the pump can provide a type of multi-level depth perception. This allows the proximity of a user's hands, for example, to the sides of the pump to control a variable setting like flow rate. For example, hands within 2 inches of the sides could represent 10 ml/hr and hands within 4 inches of the sides could represent 20 ml/hr. This type of two-handed operation serves to prevent inadvertent interactions from being sensed.

Accordingly, an infusion pump is contemplated having touchless sensors with a proximity sensor serving as a multifunction switch. Such a multifunction switch can be used for simple functions such as silencing alarms; starting, stopping and pausing infusions; and adjusting infusion delivery rate.

FIG. 13 shows a diagram of various elements of an infusion pump system 1000 including one or more proximity sensors 1011. The infusion pump 1010 can be a syringe pump as shown in FIG. 12 including a similar pump housing 1012 and shape, but is not limited to such a pump and could alternatively be a peristaltic pump or any other type of medical infusion pump having another housing and shape. The infusion pump 1010 includes a pump control system 1013, a pumping mechanism 1015, and a touchless control module 1017. The pump control system includes a processor 1019 and a memory 1021 programmable to control operation of the pumping mechanism 1015. The pumping mechanism 1015 is coupled to the pump housing in any manner including external coupling, internal coupling, or integral formation with the pump housing. As in other embodiments, the pumping mechanism 1015 generally serves to selectively urge medicament along an infusion line to a patient (or otherwise deliver medicament to the patient) and can include components specific to the type of infusion pump used. Also included in the infusion pump 1010 is the touchless control module 1017. The touchless control module 1017 generally relays commands to the pump control system 1013. The touchless control module 1017 includes at least one touchless proximity sensor 1011 configured to receive a touchless programming command for the infusion pump 1010 from a user. In some embodiments, the touchless control module 1017 may provide an alarm silencer and in others it may provide a feature with the ability to pause or restart an infusion pump 1010.

In another embodiment, an RFID sensor is used as a sensor. The RFID sensor can contain identification information for the owner of the RFID tag. This information is used to identify the individual who is interacting with the pump and provides additional authentication security for pump operation to ensure that, for example, patients, family members, and friends, do not modify pump settings without proper authorization to do so.

In addition to advantageous devices and methods discussed thus far, such as the biometric authentication features associated with FIG. 10, other related features, methods, and means for ensuring that the “five rights” are implemented, observed, and verified are contemplated. Keeping the “five rights” is important to any safety system employed in an infusion pump environment.

In general, known safety systems for infusion pumps often request confirmation of orders from clinicians though presentation of a series of questions on the pump user interfaces such as display screens on the pumps. The questions are then answered by a practitioner or user of each pump through their corresponding button presses on the user interface. If the questions are answered satisfactorily via the button inputs as determined by software logic, then the medicament order prescribed for delivery by the pump to the patient is deemed to be acceptable and the pump is permitted to operate by the associated software. However, such known systems usually do not specifically confirm an identity and authorization of the practitioner or user from whom those inputs have been received. Therefore, a system for properly identifying and authorizing or authenticating a user of an infusion pump is desired, that may also supplement verification of the “five rights”.

Accordingly, identification and authentication systems for infusion pumps are contemplated that identify and authenticate authorized practitioners or users of the pumps. Risks associated with use of the pumps by unidentified or unauthorized personnel are minimized as well. This additional capability of “five rights” verification with pumps further enhances overall patient safety.

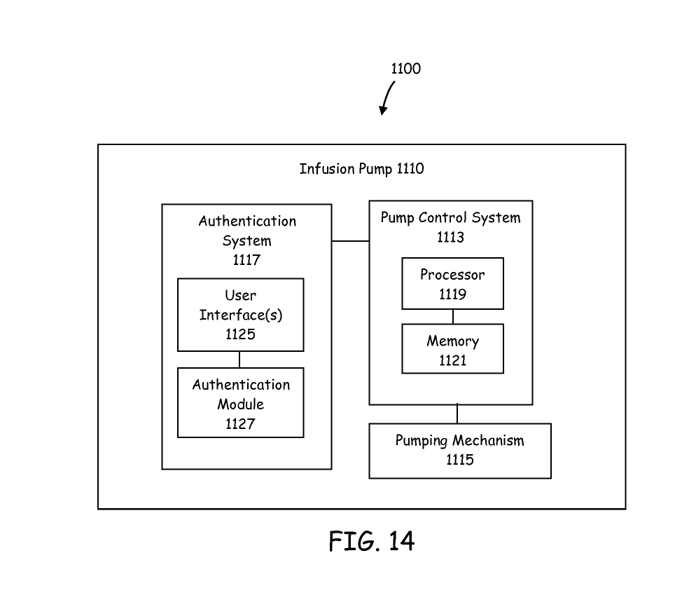

FIG. 14 shows a simple block diagram of an infusion pump system 1100 having authentication features. Specifically, the diagram illustrates an infusion pump 1110, pump control system 1113, pumping mechanism 1115, and authentication system 1117. The infusion pump 1110 can be a syringe pump, a peristaltic pump or any other type of medical infusion pump having another pump housing and shape. The pump control system 1113 includes a processor 1119 and a memory 1121 programmable to control operation of the pumping mechanism 1115. The pumping mechanism 1115 is coupled to the pump housing in any manner including external coupling, internal coupling, or integral formation with the pump housing. The pumping mechanism 1115 generally serves to selectively urge medicament along an infusion line to a patient (or otherwise deliver medicament to the patient). The authentication system 1117 of the pump includes a user interface 1125 and an authentication module 1127. The user interface 1125 contains an input mechanism that is used for recognition and logging of user identifiers that are associated with authorized users of the infusion pump. The input mechanisms of the user interface 1125 can include, but are not limited the touchless input mechanisms 30 discussed in this disclosure, touch screens, fingerprint recognition sensors, proximity sensors or other sensors recognizing gesture-based, voice-based, or audio-based inputs. Such sensing may rely on but is not limited to sensing by cameras, electric field sensors, capacitance sensors, audio sensors, proximity sensors or voice sensors. The user interface 1125 may be touch-based or touchless. User identifiers may include user signature, initials, voice, fingerprints, or identifying gestures, shapes or patterns, for example. Further user identifiers may include closely associated external objects of users having discrete capacitive signatures such as computer cards, dongles, and the like—in place of, or in combination with, recognition of names, identifying characters, and handwriting or signatures. The authentication module 1127 is used to authorize a user to control the infusion pump when user identifiers of at least one user are recognized by the authentication system as being associated with an authorized user of the pump.

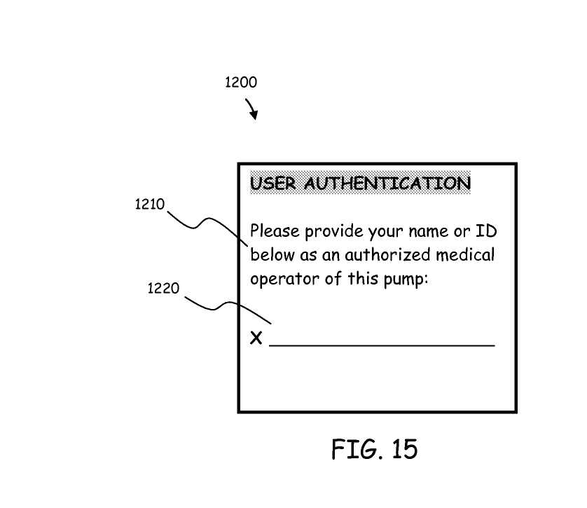

FIG. 15 shows an example of a display screen 1200 that can be used in the user interface 1125 of an infusion pump authentication system 1117. A display screen 1200 can be utilized on touch screens or touchless screens used for authorization purposes as part of an authentication system. Embodiments of the display screen are not in any way limited to the form and structure of the display screen shown in FIG. 15. Various text, shapes, layout and requested authentication actions can be requested on such a display 1200. Moreover, a user interface 1125 requesting authorization may not require a display in certain embodiments and may instead rely on voice commands or audio requests to acquire authentication information. The authorization system 1117 accordingly is configured to recognize, log and accept or reject user identifiers supplied to the authentication system.

The display screen 1200 in FIG. 15 requests an authentication in the form of a name or user ID at 1210. In response, an authorized user could sign at the line 1220 on a touchscreen or touchlessly sign in the space adjacent the pump. Many other verification marks, gestures, voice commands, or other interactions could suffice as authentication action by an authorized user. In some embodiments, user identifiers may include individual names or identifying characters of practitioners and users of the pumps rendered on the display screens. A name or other identifying character or characters can thus be required to confirm an electronic order—such as, for example, a particular infusion order that is placed on a central computer terminal in communication with a pump when the pump is commanded to start. Specifically, before the infusion pump would be allowed to have its programming modified or to start medicament delivery, an authorized name or other identifying character or characters would need to be rendered on the touch screen or touchless screen, and such input would need to be read and recognized before the system would permit the infusion pump to start delivering a medicament to the patient. Authorization of the user can happen at various times in different embodiments, and authorization of a user may happen a plurality of times throughout the programming and/or confirmation process of infusion pump programming and medicament delivery.

In addition to verifying the user controlling the infusion pump, the programming of a delivery of medicament to a patient via an infusion pump can be authenticated as well. In certain embodiments, the pump screen, or other suitable user interface display could be configured to solicit answers, from the authorized personnel, to the “five rights” questions for medication administration (i.e. Right patient? Right medication? Right dose? Right route? and Right time?). A name or identifying character or characters of an authorized person could be required after each question is answered, to further enhance safety to the patient. Such “five rights” verification could thus, advantageously, become virtually universal and automatic for health care facilities and organizations using such devices. Further, the touch screen, touchless screen or other suitable user interface working in cooperation with suitable software can also include a handwriting or signature recognition and confirmation feature, in place of or in combination with the rendering and recognition by the pump's software of the name or identifying character or characters on the user interface. Accordingly, confirming the responses to the five rights questions related to programming of the pump, by an authorized signature for example, can simultaneously serve as a verification of the user of the pump as well as the infusion programming.

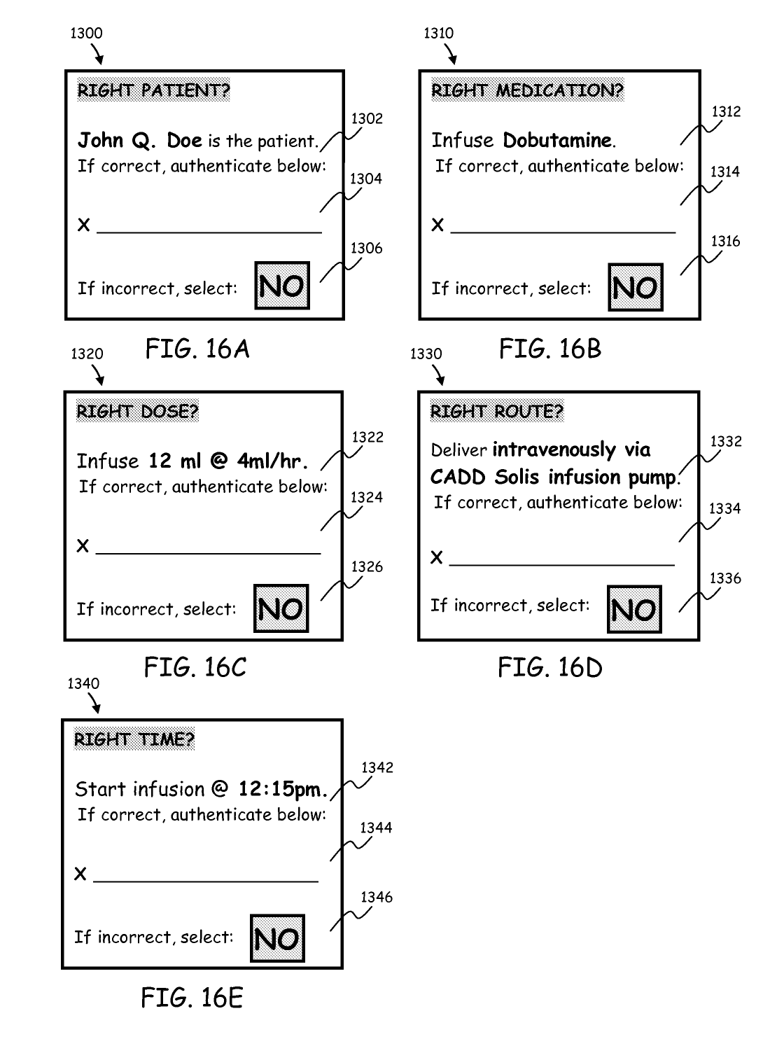

FIGS. 16A-E depict example display screens 1300, 1310, 1320, 1330, and 1340 that could be presented as part of a user interface 1125 of an authentication system 1117. These display screens can be presented sequentially in some embodiments. In other embodiments, only some of the display screens are presented or the display screens are presented in a non-sequential matter among other displays and actions of the infusion pump. The display screen 1300 relates to the five rights confirmation of “Right Patient?”. An identifying statement and confirmation request 1302 related to the patient to be infused with medicament by the infusion pump is presented. In response, an authorized user could sign at the line 1304 on a touchscreen or touchlessly sign in the space adjacent the pump. Many other verification marks, gestures, voice commands, or other interactions could suffice as authentication action by an authorized user. An option 1306 indicating that the patient name is incorrect is provided as well. If the patient is indicated as being incorrect, the error is displayed and the user is taken to a screen allowing the user to correctly identify the patient for the programmed infusion. If the patient is correct, the infusion pump operation will proceed to the next screen.

The display screen 1310 relates to the five rights confirmation of “Right medication?”. An identifying statement and confirmation request 1312 related to the medicament to be infused by the infusion pump is presented. In response, an authorized user could sign/authenticate at the line 1314 to indicate that it is correct or select option 1316 indicating that the medicament is incorrect. If the patient is indicated as being incorrect, the error is displayed and the user is taken to a screen allowing the user to correctly identify the medication for the programmed infusion. If the medicament is correct, the infusion pump operation will proceed to the next screen.

The display screen 1320 relates to the five rights confirmation of “Right dose?”. An identifying statement and confirmation request 1322 related to the dose of medicament to be infused by the infusion pump is presented. This may include the total dose delivered, the rate of delivery, or other delivery parameter or combination of dosage delivery parameters. In response, an authorized user could sign/authenticate at the line 1324 to indicate that it is correct or select option 1326 indicating that the dose of medicament is incorrect. If the dose is indicated as being incorrect, the error is displayed and the user is taken to a screen allowing the user to correctly identify the dose of medicament for the programmed infusion. If the dose is correct, the infusion pump operation will proceed to the next screen.

The display screen 1330 relates to the five rights confirmation of “Right route?”. An identifying statement and confirmation request 1332 related to the route of medicament delivery by the infusion pump is presented. This may include the method for delivering the infusion, the device used and similar delivery parameters. In response, an authorized user could sign/authenticate at the line 1334 to indicate that it is correct or select option 1336 indicating that the route of medicament is incorrect. If the route is indicated as being incorrect, the error is displayed and the user is taken to a screen allowing the user to correctly identify the route for the programmed infusion. If the route is correct, the infusion pump operation will proceed to the next screen.

The display screen 1340 relates to the five rights confirmation of “Right time?”. An identifying statement and confirmation request 1342 related to the time of medicament delivery by the infusion pump is presented. This may include the date, time of day, length of delivery or other time related confirmation. In response, an authorized user could sign/authenticate at the line 1344 to indicate that it is correct or select option 1346 indicating that the time of medicament delivery is incorrect. If the time is indicated as being incorrect, the error is displayed and the user is taken to a screen allowing the user to correctly identify the time for the programmed infusion. If the time is correct, the infusion pump operation will proceed to the next screen.

In some embodiments, after all “five rights” are confirmed, the infusion pump is authorized to proceed and operate as programmed. In some embodiments, the user of the pump is authenticated during the individual verifications of the five rights based on a user identifier as well. In some embodiments, authentication of the user will be provided by voice recognition of voice commands by an authenticated user.

The authentication system can be configured to control permission levels in use of the pumps. For example, an electronic library of authorized user names, identifying characters, and signatures could reside in each pump and/or on a central server in communication with each pump. The systems can also be configured to log usage by electronically saving or otherwise storing the names or identifying characters input to the pumps, along with other inputs associated therewith such as answers to “five rights” questions.

In an embodiment, a stylus or other similar pen-like implement could be used for inputs to the touch screen or user interface. Such an implement can also be configured to function as a key assigned to a particular authorized user, which would be required to physically interface or electronically communicate with the pump for additional security. This could be done in both touch or touchless display embodiments.

A shape, pattern, or gesture recognition component or system can be employed in an embodiment of an identification and authentication system for an infusion pump. This would be used in place of, or in combination with, recognition of names, identifying characters, and handwriting or signatures. This could be done in both touch or touchless display embodiments.

In an embodiment, a projected capacitive (“PCAP”) multi-touch component or system could be employed to identify and authenticate external objects having discrete capacitive signatures such as computer cards, dongles, and the like—in place of, or in combination with, recognition of names, identifying characters, and handwriting or signatures. The PCAP component or system could reside on or be in communication with multiple devices, whether locally or on hand-held devices or articles such as portable computers, mobile phones, patient wrist bands, patient charts or records, medicament labels, and packaging and labels for disposable medical articles such as syringes and infusion sets and manifolds, etc.

In light of the foregoing description it is therefore to be appreciated and understood that identification and authentication systems for infusion pumps, as described by example or otherwise contemplated herein, could advantageously require an authorized name or other identifying character or characters to be rendered on the touch screen or other user interface before the pump would be allowed to start; and the described “five rights” verification would further enhance patient safety. Additional benefits from such systems would result from more deliberate and thoughtful review of the pump's settings by the practitioner or user upon requiring them to input their personal identifiers. Occurrences of potentially deleterious “family controlled analgesia” or unauthorized PCA dosing would be reduced as well.

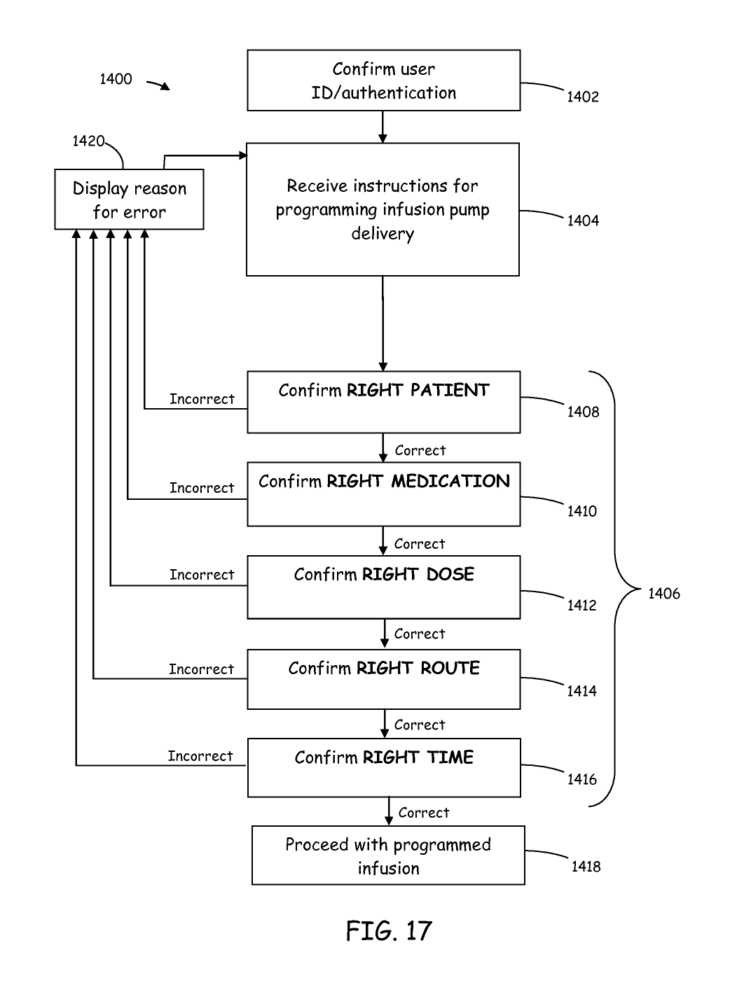

FIG. 17 is a flowchart 1400 of a method relating to a touchless programming and verification system for an infusion pump including user authentication and pump programming authentication. In general, the disclosed flowchart 1400 provides a method using authentication actions to safely and reliably controlling an infusion pump.

The method includes authenticating an authorized user of an infusion pump by confirming the user ID or user authentication at 1402. This can include both requesting a user identifier comprising information from a potential user and determining whether the potential user meets the requirements of an authorized user of the infusion pump based upon the user identifier received in response to the request. Confirmation of an authorized user may be done in response to a display screen 1200 as shown in FIG. 15 or any of various types of confirmation prompts. Such a prompt may follow other actions attempting to turn on a pump, direct or modify its programming, respond to an alarm or other action for which the user wishes to access control of the pump and its infusion programming or settings. Possible user identifiers may include, but are not limited to user signatures, initials, voice, fingerprints, or identifying gestures, shapes or patterns, for example. Further user identifiers may include closely associated external objects of users having discrete capacitive signatures such as computer cards, dongles, and the like—in place of, or in combination with, recognition of names, identifying characters, and handwriting or signatures, for example.

The method further includes receiving a programming of a delivery of medicament to a patient via the infusion pump from an authorized user at 1404. Accordingly, instructions for programming infusion pump delivery are received via one of a plurality of possible methods or types of user programming interaction. At 1406, the method includes authenticating the programming of a delivery of medicament to a patient via the infusion pump. Such authentication of programming generally includes each of the actions 1408, 1410, 1412, 1414 and 1416 discussed below. This group of actions represent confirmation of the “five rights” of medication administration.

Specifically, at 1408 the “right patient” is confirmed. This includes requesting confirmation of the patient receiving the medicament from the infusion pump and determining whether the delivery of medicament is authorized based on responses to the requested confirmation. At 1410 the “right medication” is confirmed. This includes requesting confirmation of the medicament, including medications, delivered by the infusion pump and determining whether the delivery of medicament is authorized based on responses to the requested confirmation. At 1412 the “right dose” is confirmed. This includes requesting confirmation of a dose delivered by the infusion pump and determining whether the delivery of medicament is authorized based on responses to the requested confirmation. At 1414 the “right route” is confirmed. This includes requesting confirmation of a route of delivery of the infusion pump and determining whether the delivery of medicament is authorized based on responses to the requested confirmation. At 1416 the “right time” is confirmed. This includes requesting confirmation of a time of medicament delivery by the infusion pump and determining whether the delivery of medicament is authorized based on responses to the requested confirmation. Determinations of whether one of the five rights is correct may rely, for example, on whether the user signs (on the touch screen or touchlessly) the display screen requesting confirmation of this data. See for example, the display screens in FIGS. 16A-E. If each of these rights is confirmed, the pump is enabled to proceed with the desired programmed infusion, as set forth at 1418. If any one of the “five rights” confirmations are not confirmed, the reason for the error is displayed at 1420 and the user is able to again modify and receive instructions for programming the infusion pump delivery, as at 1404 and subsequently reenter the authentication of programming at 1406.

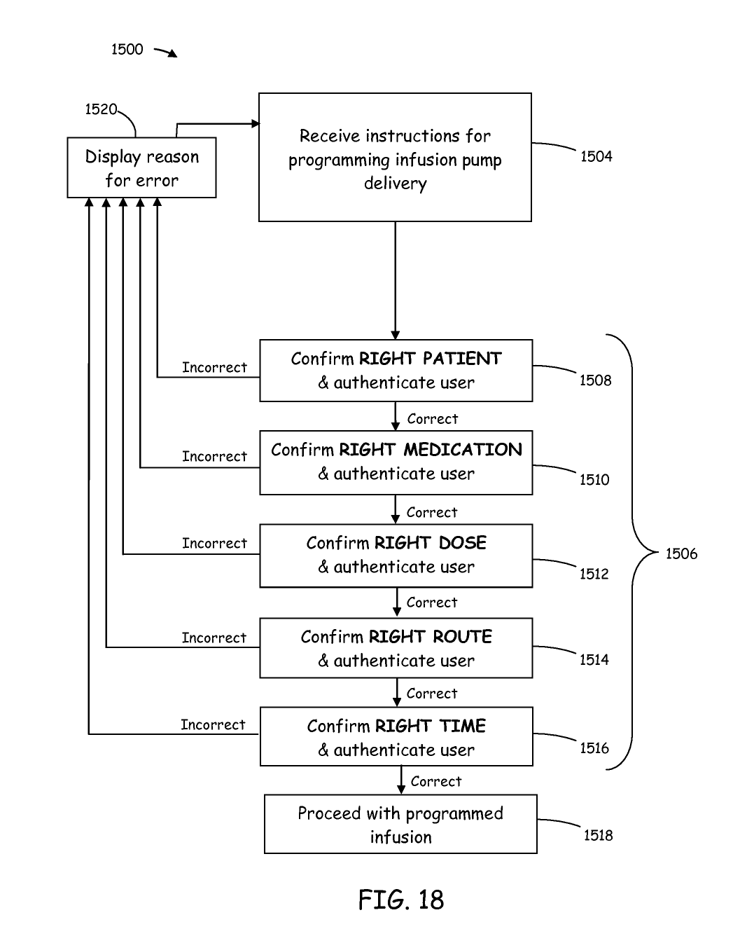

FIG. 18 is a flowchart 1500 of a method relating to a touchless programming and verification system for an infusion pump including user authentication and pump programming authentication. In general, the disclosed flowchart 1500 provides a method using authentication actions for safely and reliably controlling an infusion pump. In general, the method provided in FIG. 18 should be understood to contain similar actions to those corresponding to similar actions described in connection with FIG. 17 with a few differences. Specifically, the flowchart 1500 of the method begins with receiving a programming of a delivery of medicament to a patient via the infusion pump from an authorized user at 1504 without a prior user authentication action as at 1402. This user authentication is instead provided during the individual authentication of programming actions at 1506. At 1506, the method includes authenticating the programming of a delivery of medicament to a patient via the infusion pump. Such authentication of programming generally includes each of the actions 1508, 1510, 1512, 1514 and 1516 as similarly discussed above. This group of actions represent confirmations of the “five rights” of medication administration. In this embodiment, responses to the confirmation requests are delivered by a response, such as a user signature, which can simultaneously be used as both confirmation of the action as well as authentication of the user.

Similar to the method of FIG. 17, if each of the “rights” is confirmed, the pump is enabled to proceed with the desired programmed infusion, as set forth at 1518. If any one of the “five rights” confirmations are not confirmed, the reason for the error is displayed at 1520 and the user is able to again modify and receive instructions for programming the infusion pump delivery, as at 1504 and subsequently reenter the authentication of programming at 1506.

It should also be appreciated that the exemplary embodiment or exemplary embodiments are only examples, and are not intended to limit the scope, applicability, or configuration of the invention in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the invention as set forth in the appended claims and the legal equivalents thereof. For example, in embodiments described with a syringe-type infusion pump, it is to be understood that an ambulatory type pump can be alternatively employed.

The embodiments above are intended to be illustrative and not limiting. Additional embodiments are within the claims. Although the present invention has been described with reference to particular embodiments, workers skilled in the art will recognize that changes can be made in form and detail without departing from the spirit and scope of the invention.

Various modifications to the invention can be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

For purposes of interpreting claims herein, it is expressly intended that provisions of Title 35, United States Code, section 112, paragraph 6, are not to be invoked unless “means for” or “step for” are specifically recited in a claim.

1. An infusion pump providing safe and reliable touchless control, comprising:
a pump housing;
a pumping mechanism coupled to the pump housing that selectively delivers medicament to a patient;
a pump control system including a processor and a memory programmable to control operation of the pumping mechanism; and
a touchless control module for relaying commands to the pump control system including:
a first touchless user interface configured to receive a touchless programming command for the infusion pump from a user; and
a second touchless user interface configured to confirm the touchless programming command by receipt of a touchless confirmation command from the user.

2. The infusion pump of claim 1, wherein the first touchless user interface receives gesture-based touchless programming commands.

3. The infusion pump of claim 2, wherein the first touchless user interface includes at least one camera.

4. The infusion pump of claim 2, wherein the first touchless user interface includes an electric field sensor.

5. The infusion pump of claim 2, wherein the first touchless user interface includes a capacitance sensor.

6. The infusion pump of claim 2, wherein the gesture-based touchless programming commands are based on facial recognition and facial movements.

7. The infusion pump of claim 2, wherein the gesture-based touchless programming commands are based on finger and hand movement recognition.

8. The infusion pump of claim 2, wherein the second touchless user interface receives voice-based touchless confirmation commands.

9. The infusion pump of claim 2, wherein the second touchless user interface receives gesture-based touchless confirmation commands.

10. The infusion pump of claim 1, wherein the first touchless user interface receives touchless programming commands using optical character recognition and the second touchless user interface receives a touchless confirmation command by label recognition.

11. The infusion pump of claim 1, wherein the infusion pump includes touchless biometric user identity authentication.

12. The infusion pump of claim 1, wherein the infusion pump includes touchless biometric patient identity authentication.

13. The infusion pump of claim 11, wherein the touchless biometric user identity authentication utilizes one of: retinal scanning; facial recognition; voice recognition; voice passcode; touchless fingerprint recognition; and gesture user-specific passcode.

14. The infusion pump of claim 12, wherein the touchless biometric patient identity authentication utilizes one of: retinal scanning; facial recognition; voice recognition: voice passcode; touchless fingerprint recognition; and gestured user-specific passcode.

15. The infusion pump of claim 1, wherein the first touchless user interface is responsive to a voice-based PCA dose request of the patient.

16. An infusion pump providing safe and reliable touchless control, comprising:
voice-based touchless user interface that receives voice commands requesting one or more modifications of operating parameters of the infusion pump; and
a gesture-based touchless user interface that receives user confirmation of the one or more modifications of operating parameters of the infusion pump.

17. The infusion pump of claim 16, wherein the gesture based touchless user interface includes a camera.

18. The infusion pump of claim 16, wherein the gesture-based touchless user interface includes an electric field sensor.

19. A method of safely and reliably controlling an infusion pump in a touchless manner, comprising:
programming an infusion pump with one or more commands using a first type of touchless input in response to one or more touchless, gesture-based movements received by a touchless sensing module in the infusion pump; and
verifying the one or more commands with a second type of touchless input received by the touchless sensing module in the infusion pump.

20. The method of claim 19, wherein the second type of touchless input includes voice-based commands.

21. The method of claim 19, wherein the one or more touchless, gesture-based movements are recognized by a camera-based recognition device of the touchless sensing module.

22. An infusion pump providing safe and reliable control, comprising:
a pump housing;
a pumping mechanism coupled to the pump housing that selectively delivers medicament to a patient;
a pump control system including a processor and a memory programmable to control operation of the pumping mechanism; and
an authentication system of the infusion pump, comprising:
user interface associated with the infusion pump having an input mechanism that recognizes and logs at least one user identifier associated with is user of the infusion pump; and
an authentication module that authorizes the user to control the infusion pump upon recognition of the at least one use identifier as being associated with an authorized user of the infusion pump.

23. The infusion pump of claim 22, wherein the authentication system includes a five rights verification means.

24. The infusion pump of claim 23, wherein the input mechanism is a touch screen.

25. The infusion pump of claim 24, wherein the at least one user identifier includes a user signature.

26. The infusion pump of claim 23, wherein the user interface is touchless.

27. The infusion pump of claim 26, wherein the at least one user identifier includes a user authorization gesture.

28. A method of safely and reliably controlling an infusion pump, comprising:
authenticating an authorized user of an infusion pump, including:
requesting a user identifier comprising information from a potential user;
determining whether the potential user meets requirements of authorized users of the infusion pump based upon the user identifier received in response to the requesting;
receiving a programming of a delivery of medicament to a patient via the infusion pump from the authorized user;
authenticating the programming of the delivery of medicament to the patient via the infusion pump, including:
requesting confirmation of the patient programmed to receive medicament from the infusion pump;
requesting confirmation of the medicament, including medicaments programmed to be delivered by the infusion pump:
requesting confirmation of a dose programmed to be delivered by the infusion pump;
requesting confirmation of a route of programmed delivery of the infusion pump;
requesting confirmation of a time of programmed medicament delivery by the infusion pump; and
determining whether the programming of the delivery of medicament is authorized based on responses of the authorized user to requested confirmations.

29. The method of claim 28 wherein the user identifier is received via a touchless user interface.

30. The method of claim 28 wherein the user identifier is a user signature.

31. An infusion pump providing safe and reliable touchless control, comprising:
a pump housing;
a pumping mechanism coupled to the pump housing that selectively delivers medicament to a patient;
a pump control system including a processor and a memory programmable to control operation of the pumping mechanism; and
a touchless control module for relaying commands to the pump control system including:
a first touchless proximity sensor configured to receive a touchless command for the infusion pump from a user.

32. The infusion pump of claim 31, wherein the touchless control module includes a plurality of proximity sensors.

33. The infusion pump of claim 31, wherein the touchless control module includes an alarm silencer.

34. The infusion pump of claim 33, wherein the touchless control module includes a feature to start or pause infusion.
